(19) United States
(12) Patent Application Publication
(10) Pub. No.: US 2010/0184765 A1
(43) Pub. Date: Jul. 22, 2010

(54) Protein Kinase Inhibitors and Methods for Using Thereof

(75) Inventors: Shenlin Huang, San Diego, CA (US); Zuosheng Liu, San Diego, CA (US); Pamela A. Albaugh, San Diego, CA (US); Xing Wang, San Diego, CA (US); Shifeng Pan, San Diego, CA (US); Yongping Xie, San Diego, CA (US); Guobao Zhang, San Diego, CA (US)

Correspondence Address: NOVARTIS INSTITUTES FOR BIOMEDICAL RESEARCH, INC., 220 MASSACHUSETTS AVENUE, CAMBRIDGE, MA, 02139, US

(73) Assignee: IRM LLC

(21) Appl. No.: 12/664,765

(22) PCT Filed: Jun. 10, 2008
(86) PCT No.: PCT/US08/66426

§ 371 Date: Dec. 15, 2009

Related U.S. Application Data
(60) Provisional application No. 60/944,457, filed on Jun. 15, 2007.

(51) Int. Cl. A61K 31/5377; C07D 401/04; C07D 241/02; C07D 413/04; A61K 31/497; A61P 35/00

(52) U.S. Cl. 514/235.8; 544/328; 544/357; 544/122; 514/256; 514/252.14

(57) ABSTRACT

The invention provides compounds and pharmaceutical compositions thereof, which are useful as protein kinase inhibitors, and methods for using such compounds to treat, ameliorate or prevent a condition associated with abnormal or deregulated kinase activity. In some embodiments, the invention provides methods for using such compounds to treat, ameliorate or prevent diseases or disorders that involve abnormal activation of Alk, Abl, Aurora-A, B-Raf, C-Raf, Bcr-Abl, BRK, Blk, Bmx, BTK, C-Kit, CSK, C-Src, EphB1, EphB2, EphB4, FLT1, Fms, Flt3, Fyn, FRK3, JAK2, KDR, Lck, Lyn, PDGFRα, PDGFRβ, PKCα, SAPK2α, Src, SIK, Syk, Tie2 and TrkB kinases.

### CROSS-REFERENCE TO RELATED APPLICATIONS

This application claims the benefit of U.S. provisional application Ser. No. 60/944,457, filed Jun. 15, 2007, which is incorporated herein by reference in its entirety.

### TECHNICAL FIELD

The invention relates to protein kinase inhibitors, and methods of using such compounds.

### BACKGROUND ART

The protein kinases include a large number of family members, which play a central role in regulating a wide variety of cellular function. A partial, non-limiting, list of these kinases include: receptor tyrosine kinases such as platelet derived growth factor receptor (PDGFR), nerve growth factor receptorTrkB, C-Met, and fibroblast growth factor receptor (FGFR-3); non-receptor tyrosine kinases such as Abl and the corresponding fusion kinase Bcr-Abl, Lck, Csk, Fes, Bmx and Src; and serine/threonine kinases such as B-Raf, C-Raf, Syk, MAP kinases (e.g., MKK4, MKK6, etc.) and SAPK2α, SAPK2β and SAPK3. Aberrant kinase activity has been observed in many disease states including benign and malignant proliferative disorders, as well as diseases resulting from inappropriate activation of the immune and nervous systems. Therefore, inhibition of these kinases would have multiple therapeutic indications.

### DISCLOSURE OF THE INVENTION

The invention provides compounds and pharmaceutical compositions thereof, which may be useful as protein kinase inhibitors.

In one aspect, the invention provides compounds having Formula (1):

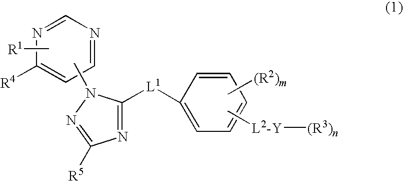

or pharmaceutically acceptable salts thereof, wherein:

L¹ is NR, NRCO or NRSO₁₋₂;

L² are independently NRCO, NRCONR, CONR, NRSO₁₋₂ or SO₁₋₂NR;

Y is a C₃₋₇ cycloalkyl, C₃₋₇ heterocycloalkyl, or a monocyclic or fused 5-10 membered aryl or heteroaryl containing N, O or S;

R¹ and R⁵ are independently H, an optionally halogenated C₁₋₆ alkyl, NR₂ or halo;

R² is an optionally halogenated C₁₋₆ alkyl or halo;

R³ is halo, substituted or unsubstituted C₁₋₆ alkyl, C₂₋₆ alkenyl, or C₂₋₆ alkynyl; optionally halogenated C₁₋₆ alkoxy, XR⁸, XO(CR₂)ₚR⁹, O(CR₂)ₚNR⁶R⁷, XNR⁶R⁷ or XNR(CR₂)ₚNR⁶R⁷;

R⁴ is NR⁶R⁷, NR(CR₂)ₚNR⁶R⁷, NRCONR⁶R⁷ or NRCO₂R⁶;

R⁶ and R⁷ are independently H, an optionally halogenated C₁₋₆ alkyl, C₂₋₆ alkenyl or C₂₋₆ alkynyl; C₁₋₆ alkanol, XR⁸ or XO(CR₂)ₚR⁹; or R⁶ and R⁷ together with N in NR⁶R⁷ may form an optionally substituted ring;

R⁸ and R⁹ are independently an optionally substituted C₃₋₇ cycloalkyl, 5-7 membered aryl, heterocyclic or heteroaryl; or R⁹ is H;

each R is H or C₁₋₆ alkyl;

each X is a bond or a C₁₋₄ alkylene;

m is 0-2; and

n and p are independently 0-4.

In the above Formula (1), L¹ may be NH. In other examples, L² is NHCO, CONH, or NHCONH. In yet other examples, R¹ may be H. In yet other examples, R² is CH₃.

In another embodiment, the compound is of Formula (2) or (3):

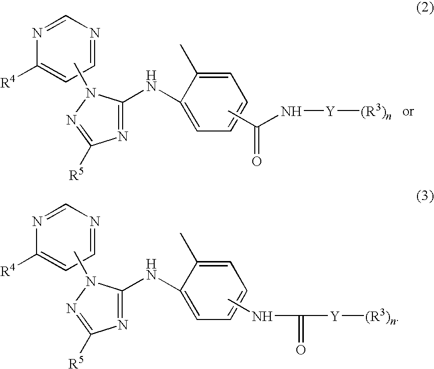

In the above Formula (1), (2) or (3), Y may be a monocyclic or fused 5-10 membered aryl or heteroaryl containing N, O or S. For example, Y may be phenyl, pyridyl, thienyl, pyrazolyl, isoxazolyl, furanyl or pyrrolyl. In other examples, R³ is halo, C₁₋₆ alkyl optionally substituted with halo, hydroxyl, alkoxy, or cyano; optionally halogenated C₁₋₆ alkoxy, XR⁸, XO(CR₂)ₚR⁹, O(CR₂)ₚNR⁶R⁷, XNR⁶R⁷ or XNR(CR₂)ₚNR⁶R⁷. For example, R⁶ and R⁷ together with N form an optionally substituted piperidinyl, morpholinyl, piperazinyl, pyrrolidinyl, pyrrolidonyl or imidazolyl. In other examples, R⁸ and R⁹ are independently an optionally substituted C₃₋₇ cycloalkyl, piperidinyl, morpholinyl, piperazinyl, pyrrolidinyl, pyrrolidonyl, imidazolyl, pyridyl, phenyl, furanyl, naphthalenyl, pyrimidinyl, triazolyl, isothiazolyl, isoxazolyl, pyrazolyl or pyrazinyl.

In the above Formula (1), (2) or (3), each optionally substituted ring is optionally substituted with halo, optionally halogenated C₁₋₆ alkyl, C₂₋₆ alkenyl, or C₂₋₆ alkynyl; nitro, cyano, XCO₁₋₂R¹⁰, XO(CR₂)ₚR¹⁰, XS(CR₂)ₚR¹⁰, XR⁸, XNR¹⁰(CR₂)ₚR¹⁰, XNR(CR₂)ₚNR₂, XNRCOR¹⁰, XNRCONR₂, XNR(CR₂)ₚOR, XNR(C═NR)NR₂, XCONR¹⁰(CR₂)ₚR¹⁰, XCONR(CR₂)ₚNR₂, XNSO₁₋₂R, XNRSR, XSO₁₋₂R⁸, XSO₁₋₂NR¹⁰(CR₂)ₚR¹⁰ or XSNR₂; wherein R¹⁰ is H, optionally halogenated C₁₋₆ alkyl, C₃₋₇ cycloalkyl, 5-7 membered aryl, heterocyclic or heteroaryl.

In another aspect, the present invention provides pharmaceutical compositions comprising a compound having Formula (1), (2) or (3), and a pharmaceutically acceptable excipient.

The invention also provides methods for modulating a protein kinase, comprising administering to a system or a subject in need thereof, a therapeutically effective amount of a compound having Formula (1), (2) or (3), or pharmaceutically acceptable salts or pharmaceutical compositions thereof, thereby modulating said protein kinase. In one embodiment, the invention provides methods for inhibiting a kinase, comprising administering to a system or a subject in need thereof, a therapeutically effective amount of a compound of Formula (1), (2) or (3).

Examples of protein kinases which may be modulated or inhibited using the compounds of the invention include but are not limited to Alk, Abl, Aurora-A, B-Raf, C-Raf, Bcr-Abl, BRK, Blk, Bmx, BTK, C-Kit, C-Src, EphB1, EphB2, EphB4, FLT1, Fms, Flt3, Fyn, FRK3, JAK2, KDR, Lck, Lyn, PDGFRα, PDGFRβ, PKCα, p38, Src, SIK, Syk, Tie2 and TrkB kinases. More particularly, the compounds of Formula (1), (2) or (3) may be used for inhibiting B-Raf.

In yet another aspect, the invention provides methods for ameliorating or treating a condition mediated by a protein kinase, such as a B-Raf-mediated condition, comprising administering to a system or subject in need of such treatment an effective amount of a compound having Formula (1), (2) or (3), or pharmaceutically acceptable salts or pharmaceutical compositions thereof, and optionally in combination with a second therapeutic agent, thereby treating said condition. For example, the compounds of the invention, optionally in combination with a chemotherapeutic agent, may be used to treat a cell proliferative disorder, including but not limited to, melanoma, leukemia, chronic myelogenous leukemia, lymphoma, osteosarcoma, or breast, renal, prostate, colorectal, thyroid, ovarian, pancreatic, neuronal, lung, uterine or gastrointestinal tumor. The compounds of the invention may also be used to treat an autoimmune disorder, including but not limited to systemic lupus erythematosus, inflammatory bowel disease, rheumatoid arthritis, or multiple sclerosis.

In the above methods for using the compounds of the invention, a compound having Formula (1), (2) or (3) may be administered to a system comprising cells or tissues. In other embodiments, a compound having Formula (1), (2) or (3) may be administered to a human or animal subject.

The invention also provides for the use of a compound of Formula (1), (2) or (3) in the manufacture of a medicament for treating a condition mediated by a protein kinase.

### DEFINITIONS

“Alkyl” refers to a moiety and as a structural element of other groups, for example halo-substituted alkyl and alkoxy, and may be straight-chained or branched. An optionally substituted alkyl, alkenyl or alkynyl as used herein may be optionally halogenated (e.g., CF₃), or may have one or more carbons that is substituted or replaced with a heteroatom, such as NR, O or S (e.g., —OCH₂CH₂O—, alkylthiol, thioalkoxy, alkylamine, etc).

“Aryl” refers to a monocyclic or fused bicyclic aromatic ring containing carbon atoms. For example, aryl may be phenyl or naphthyl. “Arylene” means a divalent radical derived from an aryl group.

“Heteroaryl” as used herein is as defined for aryl above, where one or more of the ring members are a heteroatom. Examples of heteroaryls include but are not limited to pyridyl, indolyl, indazolyl, quinoxalinyl, quinolinyl, benzofuranyl, benzopyranyl, benzothiopyranyl, benzo[1,3]dioxolyl, imidazolyl, benzoimidazolyl, pyrimidinyl, furanyl, oxazolyl, isoxazolyl, triazolyl, tetrazolyl, pyrazolyl, thienyl, etc.

A “carbocyclic ring” as used herein refers to a saturated or partially unsaturated, monocyclic, fused bicyclic or bridged polycyclic ring containing carbon atoms, which may optionally be substituted, for example, with ═O. Examples of carbocyclic rings include but are not limited to cyclopropyl, cyclobutyl, cyclopentyl, cyclohexyl, cyclopropylene, cyclohexanone, etc.

A “heterocyclic ring” as used herein is as defined for a carbocyclic ring above, wherein one or more ring carbons is a heteroatom. For example, a heterocyclic ring may contain N, O, S, —N═, —S—, —S(O), —S(O)₂—, or —NR— wherein R may be hydrogen, C₁₋₄alkyl or a protecting group. Examples of heterocyclic rings include but are not limited to morpholinyl, pyrrolidinyl, pyrrolidin-2-one, piperazinyl, piperidinyl, piperidinone, 1,4-dioxa-8-aza-spiro[4.5]dec-8-yl, etc.

The terms “co-administration” or “combined administration” or the like as used herein are meant to encompass administration of the selected therapeutic agents to a single patient, and are intended to include treatment regimens in which the agents are not necessarily administered by the same route of administration or at the same time.

The term “pharmaceutical combination” as used herein refers to a product obtained from mixing or combining active ingredients, and includes both fixed and non-fixed combinations of the active ingredients. The term “fixed combination” means that the active ingredients, e.g. a compound of Formula (1) and a co-agent, are both administered to a patient simultaneously in the form of a single entity or dosage. The term “non-fixed combination” means that the active ingredients, e.g. a compound of Formula (1) and a co-agent, are both administered to a patient as separate entities either simultaneously or sequentially with no specific time limits, wherein such administration provides therapeutically effective levels of the active ingredients in the body of the patient. The latter also applies to cocktail therapy, e.g. the administration of three or more active ingredients.

The term “therapeutically effective amount” means the amount of the subject compound that will elicit a biological or medical response in a cell, tissue, organ, system, animal or human that is being sought by the researcher, veterinarian, medical doctor or other clinician.

The term “administration” or “administering” of the subject compound means providing a compound of the invention and prodrugs thereof to a subject in need of treatment.

“Kinase Panel” is a list of kinases including but not limited to Abl, JAK2, JAK3, ALK, JNK1α1, KDR, Aurora-A, Lck, Blk, MAPK1, Bmx, MAPKAP-K2, BRK, MEK1, CaMKII, C-Met, CDK1/cyclinB, p70S6K, CHK2, PAK2, CK1, PDGFRα, CK2, PDK1, C-Kit, Pim-2, C-Raf, PKA, CSK, PKBα, Src, PKCα, DYRK2, Plk3, EGFR, ROCK-I, Fes, Ron, FGFR-3, Ros, Flt3, SAPK2α, Fms, SGK, Fyn, SIK, GSK3β, Syk, IGFR, Tie-2, IKKβ, TrkB, IR, WNK3, IRAK4, ZAP-70, ITK, AMPK, LIMK1, Rsk2, Ax1, LKB1, SAPK2β, BrSK2, Lyn, SAPK3, BTK, MAPKAP-K3, SAPK4, CaMKIV, MARK1, Snk, CDK2/cyclinA, MINK, SRPK1, CDK3/cyclinE, MKK4, TAK1, CDK5/p25, MKK6, TBK1, CDK6/cyclinD, MLCK, TrkA, CDK7/cyclinH/MAT1, MRCKβ, TSSK1, CHK1, MSK1, Yes, CK1d, MST2, ZIPK, MuSK, DAPK2, NEK2, DDR2, NEK6, DMPK, PAK4, DRAK1, PAR-1Bα, EphA1, PDGFRβ, EphA2, Pim-1, EphA5, PKBβ, EphB2, PKCβI, EphB4, PKCδ, FGFR1, PKCη, FGFR2, PKCθ, FGFR4, PKD2, Fgr, PKG1β, Flt1, PRK2, Hck, PYK2, HIPK2, Ret, IKKα, RIPK2, IRR, ROCK-II, JNK2α2, Rse, JNK3, Rsk1(h), PI3 Kγ, PI3 Kδ and PI3-Kβ.

### MODES OF CARRYING OUT THE INVENTION

The present invention provides compounds and pharmaceutical compositions thereof, which may be useful as protein kinase inhibitors.

In one aspect, the invention provides compounds having Formula (1):

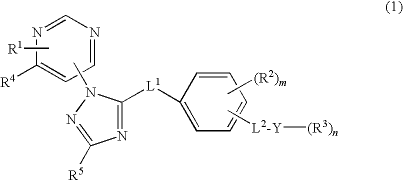

or pharmaceutically acceptable salts thereof, wherein:

L¹ is NR, NRCO or NRSO₁₋₂;

L² are independently NRCO, NRCONR, CONR, NRSO₁₋₂ or SO₁₋₂NR;

Y is a C₃₋₇ cycloalkyl, C₃₋₇ heterocycloalkyl, or a monocyclic or fused 5-10 membered aryl or heteroaryl containing N, O or S;

R¹ and R⁵ are independently H, an optionally halogenated C₁₋₆ alkyl, NR₂ or halo;

R² is an optionally halogenated C₁₋₆ alkyl or halo;

R³ is halo, substituted or unsubstituted C₁₋₆ alkyl, C₂₋₆ alkenyl, or C₂₋₆ alkynyl; optionally halogenated C₁₋₆ alkoxy, XR⁸, XO(CR₂)ₚR⁹, O(CR₂)ₚNR⁶R⁷, XNR⁶R⁷ or XNR(CR₂)ₚNR⁶R⁷;

R⁴ is NR⁶R⁷, NR(CR₂)ₚNR⁶R⁷, NRCONR⁶R⁷ or NRCO₂R⁶;

R⁶ and R⁷ are independently H, an optionally halogenated C₁₋₆ alkyl, C₂₋₆ alkenyl or C₂₋₆ alkynyl; C₁₋₆ alkanol, XR⁸ or XO(CR₂)ₚR⁹; or R⁶ and R⁷ together with N in NR⁶R⁷ may form an optionally substituted ring;

R⁸ and R⁹ are independently an optionally substituted C₃₋₇ cycloalkyl, 5-7 membered aryl, heterocyclic or heteroaryl; or R⁹ is H;

each R is H or C₁₋₆ alkyl;

each X is a bond or a C₁₋₄ alkylene;

m is 0-2; and

n and p are independently 0-4.

In one embodiment, the compound is of Formula (2) or (3):

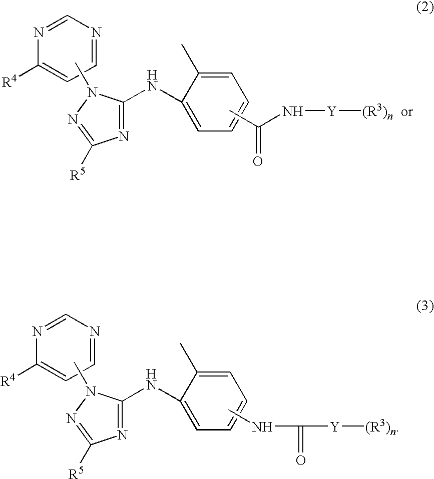

Representative compounds of the invention include but are not limited to:
N-{4-Methyl-3-[2-(6-methylamino-pyrimidin-4-yl)-2H-[1,2,4]triazol-3-ylamino]-phenyl}-3-trifluoromethyl-benzamide;
4-Methyl-3-[2-(6-methylamino-pyrimidin-4-yl)-2H-[1,2,4]triazol-3-ylamino]-N-(3-trifluoromethyl-phenyl)-benzamide;
4-Methyl-3-(3-methyl-1-(6-(methylamino)pyrimidin-4-yl)-1H-1,2,4-triazol-5-ylamino)-N-(3-(4-methylpiperazin-1-yl)-5-(trifluoromethyl)phenyl)benzamide;
1-{4-Methyl-3-[2-(6-methylamino-pyrimidin-4-yl)-2H-[1,2,4]triazol-3-ylamino]-phenyl}-3-(3-pyrrolidin-1-ylmethyl-5-trifluoromethyl-phenyl)-urea;
3-(1,1-difluoroethyl)-N-(4-methyl-3-(1-(6-(methylamino)pyrimidin-4-yl)-1H-1,2,4-triazol-5-ylamino)phenyl)benzamide;
2-tert-butyl-N-(4-methyl-3-(1-(6-(methylamino)pyrimidin-4-yl)-1H-1,2,4-triazol-5-ylamino)phenyl)isonicotinamide;
2-(2-hydroxypropan-2-yl)-N-(4-methyl-3-(1-(6-(methylamino)pyrimidin-4-yl)-1H-1,2,4-triazol-5-ylamino)phenyl)isonicotinamide;
2-(1,1-difluoroethyl)-N-(4-methyl-3-(1-(6-(methylamino)pyrimidin-4-yl)-1H-1,2,4-triazol-5-ylamino)phenyl)isonicotinamide;
3-(4-ethylpiperazin-1-yl)-N-(4-methyl-3-(1-(6-(methylamino)pyrimidin-4-yl)-1H-1,2,4-triazol-5-ylamino)phenyl)-5-(trifluoromethyl)benzamide;
3-isopropoxy-N-(4-methyl-3-(1-(6-(methylamino)pyrimidin-4-yl)-1H-1,2,4-triazol-5-ylamino)phenyl)benzamide;
N-(4-methyl-3-(1-(6-(methylamino)pyrimidin-4-yl)-1H-1,2,4-triazol-5-ylamino)phenyl)-3-(trifluoromethoxy)benzamide;
3-(2-cyanobutan-2-yl)-N-(4-methyl-3-(1-(6-(methylamino)pyrimidin-4-yl)-1H-1,2,4-triazol-5-ylamino)phenyl)benzamide;
3-(2-cyanopropan-2-yl)-N-(4-methyl-3-(1-(6-(methylamino)pyrimidin-4-yl)-1H-1,2,4-triazol-5-ylamino)phenyl)benzamide;
3-(2-hydroxypropan-2-yl)-N-(4-methyl-3-(1-(6-(methylamino)pyrimidin-4-yl)-1H-1,2,4-triazol-5-ylamino)phenyl)benzamide;
N-(4-methyl-3-(1-(6-(methylamino)pyrimidin-4-yl)-1H-1,2,4-triazol-5-ylamino)phenyl)-3-(4-methylpiperazin-1-yl)-5-(trifluoromethyl)benzamide;
N-(4-methyl-3-(1-(6-(methylamino)pyrimidin-4-yl)-1H-1,2,4-triazol-5-ylamino)phenyl)-3-(piperazin-1-yl)-5-(trifluoromethyl)benzamide;
N-(4-methyl-3-(1-(6-(methylamino)pyrimidin-4-yl)-1H-1,2,4-triazol-5-ylamino)phenyl)-3-(1-methylpiperidin-4-yloxy)-5-(trifluoromethyl)benzamide;
3-(4-(2-hydroxyethyl)piperazin-1-yl)-N-(4-methyl-3-(1-(6-(methylamino)pyrimidin-4-yl)-1H-1,2,4-triazol-5-ylamino)phenyl)-5-(trifluoromethyl)benzamide;
N-(4-methyl-3-(1-(6-(methylamino)pyrimidin-4-yl)-1H-1,2,4-triazol-5-ylamino)phenyl)-3-(piperidin-4-yloxy)-5-(trifluoromethyl)benzamide;
N-(3-(1-(6-(2,3-dihydroxypropylamino)pyrimidin-4-yl)-1H-1,2,4-triazol-5-ylamino)-4-methylphenyl)-3-(trifluoromethyl)benzamide;
4-fluoro-N-(4-methyl-3-(1-(6-(methylamino)pyrimidin-4-yl)-1H-1,2,4-triazol-5-ylamino)phenyl)-3-(trifluoromethyl)benzamide;
3-fluoro-N-(4-methyl-3-(1-(6-(methylamino)pyrimidin-4-yl)-1H-1,2,4-triazol-5-ylamino)phenyl)-5-(trifluoromethyl)benzamide;
4-chloro-N-(4-methyl-3-(1-(6-(methylamino)pyrimidin-4-yl)-1H-1,2,4-triazol-5-ylamino)phenyl)-3-(trifluoromethyl)benzamide;
4-methyl-3-(1-(6-(methylamino)pyrimidin-4-yl)-1H-1,2,4-triazol-5-ylamino)-N-(3-(4-methylpiperazin-1-yl)-5-(trifluoromethyl)phenyl)benzamide;
N-(3-tert-butylphenyl)-4-methyl-3-(1-(6-(methylamino)pyrimidin-4-yl)-1H-1,2,4-triazol-5-ylamino)benzamide;
3-(1-(6-(cyclopropylamino)pyrimidin-4-yl)-1H-1,2,4-triazol-5-ylamino)-4-methyl-N-(3-(4-methylpiperazin-1-yl)-5-(trifluoromethyl)phenyl)benzamide;
3-(1-(6-(cyclopropylamino)pyrimidin-4-yl)-1H-1,2,4-triazol-5-ylamino)-N-(3-(4-ethylpiperazin-1-yl)-5-(trifluoromethyl)phenyl)-4-methylbenzamide;
N-(3-(4-ethylpiperazin-1-yl)-5-(trifluoromethyl)phenyl)-4-methyl-3-(1-(6-(methylamino)pyrimidin-4-yl)-1H-1,2,4-triazol-5-ylamino)benzamide;
3-(1-(6-aminopyrimidin-4-yl)-1H-1,2,4-triazol-5-ylamino)-4-methyl-N-(3-(4-methylpiperazin-1-yl)-5-(trifluoromethyl)phenyl)benzamide;
3-(1-(6-(methoxyamino)pyrimidin-4-yl)-1H-1,2,4-triazol-5-ylamino)-4-methyl-N-(3-(4-methylpiperazin-1-yl)-5-(trifluoromethyl)phenyl)benzamide;
N-(3-(4-ethylpiperazin-1-yl)-5-(trifluoromethyl)phenyl)-3-(1-(6-(methoxyamino)pyrimidin-4-yl)-1H-1,2,4-triazol-5-ylamino)-4-methylbenzamide;
3-(1-(6-aminopyrimidin-4-yl)-1H-1,2,4-triazol-5-ylamino)-N-(3-(4-ethylpiperazin-1-yl)-5-(trifluoromethyl)phenyl)-4-methylbenzamide;
3-(1-(6-(cyclopropylamino)pyrimidin-4-yl)-1H-1,2,4-triazol-5-ylamino)-N-(3-(4-isopropylpiperazin-1-yl)-5-(trifluoromethyl)phenyl)-4-methylbenzamide;
N-(3-(4-isopropylpiperazin-1-yl)-5-(trifluoromethyl)phenyl)-4-methyl-3-(1-(6-(methylamino)pyrimidin-4-yl)-1H-1,2,4-triazol-5-ylamino)benzamide;
N-(3-(4-isopropylpiperazin-1-yl)-5-(trifluoromethyl)phenyl)-3-(1-(6-(methoxyamino)pyrimidin-4-yl)-1H-1,2,4-triazol-5-ylamino)-4-methylbenzamide;
N-(2-tert-butylpyridin-4-yl)-4-methyl-3-(1-(6-(methylamino)pyrimidin-4-yl)-1H-1,2,4-triazol-5-ylamino)benzamide;
4-methyl-3-(1-(6-(methylamino)pyrimidin-4-yl)-1H-1,2,4-triazol-5-ylamino)-N-(4-(trifluoromethyl)pyridin-2-yl)benzamide;
N-(3-(3-hydroxycyclobutyl)-5-(trifluoromethyl)phenyl)-4-methyl-3-(1-(6-(methylamino)pyrimidin-4-yl)-1H-1,2,4-triazol-5-ylamino)benzamide;
2-methoxy-N-(4-methyl-3-(1-(6-(methylamino)pyrimidin-4-yl)-1H-1,2,4-triazol-5-ylamino)phenyl)-6-(trifluoromethyl)isonicotinamide;
4-methyl-3-(3-methyl-1-(6-(methylamino)pyrimidin-4-yl)-1H-1,2,4-triazol-5-ylamino)-N-(3-(trifluoromethyl)phenyl)benzamide;
N-(3-(4-hydroxypiperidin-1-yl)-5-(trifluoromethyl)phenyl)-4-methyl-3-(3-methyl-1-(6-(methylamino)pyrimidin-4-yl)-1H-1,2,4-triazol-5-ylamino)benzamide;
3-(1,1-difluoroethyl)-N-(4-methyl-3-(3-methyl-1-(6-(methylamino)pyrimidin-4-yl)-1H-1,2,4-triazol-5-ylamino)phenyl)benzamide;
N-(4-methyl-3-(1-(6-(2-(pyrrolidin-1-yl)ethylamino)pyrimidin-4-yl)-1H-1,2,4-triazol-5-ylamino)phenyl)-3-(trifluoromethyl)benzamide;
N-(2-tert-butylpyridin-4-yl)-4-methyl-3-(1-(6-(2-(pyrrolidin-1-yl)ethylamino)pyrimidin-4-yl)-1H-1,2,4-triazol-5-ylamino)benzamide;
N-(3-(1-(6-(2-(dimethylamino)ethylamino)pyrimidin-4-yl)-1H-1,2,4-triazol-5-ylamino)-4-methylphenyl)-3-(trifluoromethyl)benzamide;
N-(2-tert-butylpyridin-4-yl)-3-(1-(6-(2-(dimethylamino)ethylamino)pyrimidin-4-yl)-1H-1,2,4-triazol-5-ylamino)-4-methylbenzamide;
4-methyl-3-(1-(6-(3-(4-methylpiperazin-1-yl)propylamino)pyrimidin-4-yl)-1H-1,2,4-triazol-5-ylamino)-N-(3-(trifluoromethyl)phenyl)benzamide;
N-(2-tert-butylpyridin-4-yl)-4-methyl-3-(1-(6-(3-(4-methylpiperazin-1-yl)propylamino)pyrimidin-4-yl)-1H-1,2,4-triazol-5-ylamino)benzamide;
4-methyl-3-(1-(6-(3-morpholinopropylamino)pyrimidin-4-yl)-1H-1,2,4-triazol-5-ylamino)-N-(3-(trifluoromethyl)phenyl)benzamide;
N-(2-tert-butylpyridin-4-yl)-4-methyl-3-(1-(6-(3-morpholinopropylamino)pyrimidin-4-yl)-1H-1,2,4-triazol-5-ylamino)benzamide;
4-methyl-3-(1-(6-(1-methylpiperidin-4-ylamino)pyrimidin-4-yl)-1H-1,2,4-triazol-5-ylamino)-N-(3-(trifluoromethyl)phenyl)benzamide;
3-(1-(6-(cyclopropylamino)pyrimidin-4-yl)-1H-1,2,4-triazol-5-ylamino)-4-methyl-N-(3-(trifluoromethyl)phenyl)benzamide;
N-(2-tert-butylpyridin-4-yl)-3-(1-(6-(cyclopropylamino)pyrimidin-4-yl)-1H-1,2,4-triazol-5-ylamino)-4-methylbenzamide;
3-(1-(6-(2-methoxyethylamino)pyrimidin-4-yl)-1H-1,2,4-triazol-5-ylamino)-4-methyl-N-(3-(trifluoromethyl)phenyl)benzamide;
N-(2-tert-butylpyridin-4-yl)-3-(1-(6-(2-methoxyethylamino)pyrimidin-4-yl)-1H-1,2,4-triazol-5-ylamino)-4-methylbenzamide;
N-(2-tert-butylpyridin-4-yl)-4-methyl-3-(1-(6-(methylamino)pyrimidin-4-yl)-1H-1,2,4-triazol-5-ylamino)benzamide;
4-methyl-3-(1-(6-(methylamino)pyrimidin-4-yl)-1H-1,2,4-triazol-5-ylamino)-N-(4-(trifluoromethyl)pyridin-2-yl)benzamide;
N-(4-methyl-3-(1-(6-(4-methylpiperazin-1-ylamino)pyrimidin-4-yl)-1H-1,2,4-triazol-5-ylamino)phenyl)-3-(trifluoromethyl)benzamide;
N-(2-tert-butylpyridin-4-yl)-4-methyl-3-(1-(6-(4-methylpiperazin-1-ylamino)pyrimidin-4-yl)-1H-1,2,4-triazol-5-ylamino)benzamide;
N-(4-methyl-3-(1-(6-(2-morpholinoethylamino)pyrimidin-4-yl)-1H-1,2,4-triazol-5-ylamino)phenyl)-3-(trifluoromethyl)benzamide;
N-(2-tert-butylpyridin-4-yl)-4-methyl-3-(1-(6-(2-morpholinoethylamino)pyrimidin-4-yl)-1H-1,2,4-triazol-5-ylamino)benzamide;
4-methyl-3-(1-(6-(4-methylpiperazin-1-ylamino)pyrimidin-4-yl)-1H-1,2,4-triazol-5-ylamino)-N-(4-(trifluoromethyl)pyridin-2-yl)benzamide;
4-methyl-3-(1-(6-(2-morpholinoethylamino)pyrimidin-4-yl)-1H-1,2,4-triazol-5-ylamino)-N-(4-(trifluoromethyl)pyridin-2-yl)benzamide;
4-methyl-3-(1-(6-(4-methylpiperazin-1-ylamino)pyrimidin-4-yl)-1H-1,2,4-triazol-5-ylamino)-N-(3-(trifluoromethyl)phenyl)benzamide;
4-methyl-3-(1-(6-(2-morpholinoethylamino)pyrimidin-4-yl)-1H-1,2,4-triazol-5-ylamino)-N-(3-(trifluoromethyl)phenyl)benzamide;
2-fluoro-N-(4-methyl-3-(1-(6-(4-methylpiperazin-1-ylamino)pyrimidin-4-yl)-1H-1,2,4-triazol-5-ylamino)phenyl)-5-(trifluoromethyl)benzamide;
3-fluoro-N-(4-methyl-3-(1-(6-(4-methylpiperazin-1-ylamino)pyrimidin-4-yl)-1H-1,2,4-triazol-5-ylamino)phenyl)-5-(trifluoromethyl)benzamide;
4-fluoro-N-(4-methyl-3-(1-(6-(4-methylpiperazin-1-ylamino)pyrimidin-4-yl)-1H-1,2,4-triazol-5-ylamino)phenyl)-3-(trifluoromethyl)benzamide;
4-chloro-N-(4-methyl-3-(1-(6-(4-methylpiperazin-1-ylamino)pyrimidin-4-yl)-1H-1,2,4-triazol-5-ylamino)phenyl)-3-(trifluoromethyl)benzamide;
2-tert-butyl-N-(4-methyl-3-(1-(6-(4-methylpiperazin-1-ylamino)pyrimidin-4-yl)-1H-1,2,4-triazol-5-ylamino)phenyl)isonicotinamide;
2-(1,1-difluoroethyl)-N-(4-methyl-3-(1-(6-(4-methylpiperazin-1-ylamino)pyrimidin-4-yl)-1H-1,2,4-triazol-5-ylamino)phenyl)isonicotinamide;
3-(2-cyanopropan-2-yl)-N-(4-methyl-3-(1-(6-(4-methylpiperazin-1-ylamino)pyrimidin-4-yl)-1H-1,2,4-triazol-5-ylamino)phenyl)benzamide;
3-(2-methoxypropan-2-yl)-N-(4-methyl-3-(1-(6-(4-methylpiperazin-1-ylamino)pyrimidin-4-yl)-1H-1,2,4-triazol-5-ylamino)phenyl)benzamide;
2-fluoro-N-(4-methyl-3-(1-(6-(2-morpholinoethylamino)pyrimidin-4-yl)-1H-1,2,4-triazol-5-ylamino)phenyl)-5-(trifluoromethyl)benzamide;
3-fluoro-N-(4-methyl-3-(1-(6-(2-morpholinoethylamino)pyrimidin-4-yl)-1H-1,2,4-triazol-5-ylamino)phenyl)-5-(trifluoromethyl)benzamide;
4-fluoro-N-(4-methyl-3-(1-(6-(2-morpholinoethylamino)pyrimidin-4-yl)-1H-1,2,4-triazol-5-ylamino)phenyl)-3-(trifluoromethyl)benzamide;
4-chloro-N-(4-methyl-3-(1-(6-(2-morpholinoethylamino)pyrimidin-4-yl)-1H-1,2,4-triazol-5-ylamino)phenyl)-3-(trifluoromethyl)benzamide;
2-tert-butyl-N-(4-methyl-3-(1-(6-(2-morpholinoethylamino)pyrimidin-4-yl)-1H-1,2,4-triazol-5-ylamino)phenyl)isonicotinamide;
2-(1,1-difluoroethyl)-N-(4-methyl-3-(1-(6-(2-morpholinoethylamino)pyrimidin-4-yl)-1H-1,2,4-triazol-5-ylamino)phenyl)isonicotinamide;
3-(2-cyanopropan-2-yl)-N-(4-methyl-3-(1-(6-(2-morpholinoethylamino)pyrimidin-4-yl)-1H-1,2,4-triazol-5-ylamino)phenyl)benzamide;
3-(2-methoxypropan-2-yl)-N-(4-methyl-3-(1-(6-(2-morphohnoethylamino)pyrimidin-4-yl)-1H-1,2,4-triazol-5-ylamino)phenyl)benzamide;
3-(4-ethylpiperazin-1-yl)-N-(4-methyl-3-(1-(6-(2-morphohnoethylamino)pyrimidin-4-3-yl)-1H-1,2,4-triazol-5-ylamino)phenyl)-5-(trifluoromethyl)benzamide;
tert-butyl 4-(3-(4-methyl-3-(1-(6-(2-morphohnoethylamino)pyrimidin-4-yl)-1H-1,2,4-triazol-5-ylamino)phenylcarbamoyl)-5-(trifluoromethyl)phenyl)piperazine-1-carboxylate;
N-(4-methyl-3-(1-(6-(morphohnoamino)pyrimidin-4-yl)-1H-1,2,4-triazol-5-ylamino)phenyl)-3-(trifluoromethyl)benzamide;
4-methyl-3-(1-(6-(morphohnoamino)pyrimidin-4-yl)-1H-1,2,4-triazol-5-ylamino)-N-(3-(trifluoromethyl)phenyl)benzamide;
N-(4-methyl-3-(1-(6-(2-(4-methylpiperazin-1-yl)ethylamino)pyrimidin-4-yl)-1H-1,2,4-triazol-5-ylamino)phenyl)-3-(trifluoromethyl)benzamide;
4-methyl-3-(1-(6-(2-(4-methylpiperazin-1-yl)ethylamino)pyrimidin-4-yl)-1H-1,2,4-triazol-5-ylamino)-N-(3-(trifluoromethyl)phenyl)benzamide;
N-(4-methyl-3-(1-(6-(2-morphohnoethylamino)pyrimidin-4-yl)-1H-1,2,4-triazol-5-ylamino)phenyl)-3-(piperazin-1-yl)-5-(trifluoromethyl)benzamide;
1-(4-chloro-3-(trifluoromethyl)phenyl)-3-(4-methyl-3-(1-(6-(4-methylpiperazin-1-ylamino)pyrimidin-4-yl)-1H-1,2,4-triazol-5-ylamino)phenyl)urea;
1-(4-fluoro-3-(trifluoromethyl)phenyl)-3-(4-methyl-3-(1-(6-(4-methylpiperazin-1-ylamino)pyrimidin-4-yl)-1H-1,2,4-triazol-5-ylamino)phenyl)urea;
N-(4-chloro-3-(trifluoromethyl)phenyl)-4-methyl-3-(1-(6-(2-morphohnoethylamino)pyrimidin-4-yl)-1H-1,2,4-triazol-5-ylamino)benzamide;
N-(4-fluoro-3-(trifluoromethyl)phenyl)-4-methyl-3-(1-(6-(2-morphohnoethylamino)pyrimidin-4-yl)-1H-1,2,4-triazol-5-ylamino)benzamide;
N-(4-methyl-3-(3-(methylamino)-1-(6-(methylamino)pyrimidin-4-yl)-1H-1,2,4-triazol-5-ylamino)phenyl)-3-(trifluoromethyl)benzamide;
1-(4-methyl-3-(1-(6-(methylamino)pyrimidin-4-yl)-1H-1,2,4-triazol-5-ylamino)phenyl)-3-(3-(trifluoromethyl)phenyl)urea;
1-(3-fluoro-5-(trifluoromethyl)phenyl)-3-(4-methyl-3-(1-(6-(methylamino)pyrimidin-4-yl)-1H-1,2,4-triazol-5-ylamino)phenyl)urea;
1-(4-fluoro-3-(trifluoromethyl)phenyl)-3-(4-methyl-3-(1-(6-(methylamino)pyrimidin-4-yl)-1H-1,2,4-triazol-5-ylamino)phenyl)urea;
1-(4-chloro-3-(trifluoromethyl)phenyl)-3-(4-methyl-3-(1-(6-(methylamino)pyrimidin-4-yl)-1H-1,2,4-triazol-5-ylamino)phenyl)urea;
1-(3-(4-ethylpiperazin-1-yl)-5-(trifluoromethyl)phenyl)-3-(4-methyl-3-(1-(6-(methylamino)pyrimidin-4-yl)-1H-1,2,4-triazol-5-ylamino)phenyl)urea;
1-(4-methyl-3-(1-(6-(methylamino)pyrimidin-4-yl)-1H-1,2,4-triazol-5-ylamino)phenyl)-3-(4-((4-methylpiperazin-1-yl)methyl)-3-(trifluoromethyl)phenyl)urea;
N-(3-(2-(dimethylamino)ethoxy)-5-(trifluoromethyl)phenyl)-4-methyl-3-(1-(6-(methylamino)pyrimidin-4-yl)-1H-1,2,4-triazol-5-ylamino)benzamide;
N-(3-(3-(dimethylamino)propoxy)-5-(trifluoromethyl)phenyl)-4-methyl-3-(1-(6-(methylamino)pyrimidin-4-yl)-1H-1,2,4-triazol-5-ylamino)benzamide;
4-methyl-3-(1-(6-(methylamino)pyrimidin-4-yl)-1H-1,2,4-triazol-5-ylamino)-N-(3-(2-(2-oxopyrrolidin-1-yl)ethoxy)-5-(trifluoromethyl)phenyl)benzamide;
4-methyl-3-(1-(6-(methylamino)pyrimidin-4-yl)-1H-1,2,4-triazol-5-ylamino)-N-(3-(2-(pyrrolidin-1-yl)ethoxy)-5-(trifluoromethyl)phenyl)benzamide;
N-(3-(2-(diethylamino)ethoxy)-5-(trifluoromethyl)phenyl)-4-methyl-3-(1-(6-(methylamino)pyrimidin-4-yl)-1H-1,2,4-triazol-5-ylamino)benzamide;
1-(2-fluoro-5-(trifluoromethyl)phenyl)-3-(4-methyl-3-(1-(6-(methylamino)pyrimidin-4-yl)-1H-1,2,4-triazol-5-ylamino)phenyl)urea;
1-(2-chloro-5-(trifluoromethyl)phenyl)-3-(4-methyl-3-(1-(6-(methylamino)pyrimidin-4-yl)-1H-1,2,4-triazol-5-ylamino)phenyl)urea;
1-(3-(4-ethylpiperazin-1-yl)-5-(trifluoromethyl)phenyl)-3-(4-methyl-3-(1-(6-(methylamino)pyrimidin-4-yl)-1H-1,2,4-triazol-5-ylamino)phenyl)urea;
1-(3-((2-(dimethylamino)ethyl)(methyl)amino)-5-(trifluoromethyl)phenyl)-3-(4-methyl-3-(1-(6-(methylamino)pyrimidin-4-yl)-1H-1,2,4-triazol-5-ylamino)phenyl)urea;
1-(3-((2-methoxyethyl)(methyl)amino)-5-(trifluoromethyl)phenyl)-3-(4-methyl-3-(1-(6-(methylamino)pyrimidin-4-yl)-1H-1,2,4-triazol-5-ylamino)phenyl)urea;
1-(3-(2-(dimethylamino)ethoxy)-5-(trifluoromethyl)phenyl)-3-(4-methyl-3-(1-(6-(methylamino)pyrimidin-4-yl)-1H-1,2,4-triazol-5-ylamino)phenyl)urea;
1-(3-((4-ethylpiperazin-1-yl)methyl)-5-(trifluoromethyl)phenyl)-3-(4-methyl-3-(1-(6-(methylamino)pyrimidin-4-yl)-1H-1,2,4-triazol-5-ylamino)phenyl)urea;
N-(3-((dimethylamino)methyl)-5-(trifluoromethyl)phenyl)-4-methyl-3-(1-(6-(methylamino)pyrimidin-4-yl)-1H-1,2,4-triazol-5-ylamino)benzamide; or
3-((3-hydroxyazetidin-1-yl)methyl)-N-(4-methyl-3-(1-(6-(methylamino)pyrimidin-4-yl)-1H-1,2,4-triazol-5-ylamino)phenyl)-5-(trifluoromethyl)benzamide, or

pharmaceutically acceptable salts thereof.

Compounds having Formula (1), (2) or (3) may be useful as protein kinase inhibitors. For example, compounds having Formula (1), (2) or (3), and pharmaceutically acceptable salts, solvates, N-oxides, prodrugs and isomers thereof, may be used for the treatment of a kinase-mediated condition or disease, such as diseases mediated by Alk, Abl, Aurora-A, B-Raf, C-Raf, Bcr-Abl, BRK, Blk, Bmx, BTK, C-Kit, C-Src, EphB1, EphB2, EphB4, FLT1, Fms, Flt3, Fyn, JAK2, KDR, Lck, Lyn, PDGFRα, PDGFRβ, PKCα, p38 (p38 MAP kinase, SAPK2α), Src, SIK, Syk, Tie2 and TrkB kinases, or a combination thereof.

The compounds of the invention may also be used in combination with a second therapeutic agent, for ameliorating a condition mediated by a protein kinase, such as a B-Raf-mediated condition. For example, the compounds of the invention may be used in combination with a chemotherapeutic agent to treat a cell proliferative disorder, including but not limited to, lymphoma, osteosarcoma, melanoma, or breast, renal, prostate, colorectal (colon), thyroid, ovarian, pancreatic, neuronal, lung, uterine, gastrointestinal tumor or cholangiocarcinoma. In particular embodiments, the compounds of the invention may be used to treat melanoma, thyroid cancer, colon cancer, cholangiocarcinoma or ovarian cancer. (See e.g., Davies et al., Nature 417:949-54 (2002); Brose et al., Cancer Res. 62:6997-7000 (2002); Tuveson et al., Cancer Cell 4:95-8 (2003); Karasides et al., Oncogene 23:6292-8 (2004)).

Examples of chemotherapeutic agents which may be used in the compositions and methods of the invention include but are not limited to anthracyclines, alkylating agents (e.g., mitomycin C), alkyl sulfonates, aziridines, ethylenimines, methylmelamines, nitrogen mustards, nitrosoureas, antibiotics, antimetabolites, folic acid analogs (e.g., dihydrofolate reductase inhibitors such as methotrexate), purine analogs, pyrimidine analogs, enzymes, podophyllotoxins, platinum-containing agents, interferons, and interleukins. Particular examples of known chemotherapeutic agents which may be used in the compositions and methods of the invention include, but are not limited to, busulfan, improsulfan, piposulfan, benzodepa, carboquone, meturedepa, uredepa, altretamine, triethylenemelamine, triethylenephosphoramide, triethylenethiophosphoramide, trimethylolomelamine, chlorambucil, chlomaphazine, cyclophosphamide, estramustine, ifosfamide, mechlorethamine, mechlorethamine oxide hydrochloride, melphalan, novembichin, phenesterine, prednimustine, trofosfamide, uracil mustard, carmustine, chlorozotocin, fotemustine, lomustine, nimustine, ranimustine, dacarbazine, mannomustine, mitobronitol, mitolactol, pipobroman, aclacinomycins, actinomycin F(1), anthramycin, azaserine, bleomycin, cactinomycin, carubicin, carzinophilin, chromomycin, dactinomycin, daunorubicin, daunomycin, 6-diazo-5-oxo-1-norleucine, doxorubicin, epirubicin, mitomycin C, mycophenolic acid, nogalamycin, olivomycin, peplomycin, plicamycin, porfiromycin, puromycin, streptonigrin, streptozocin, tubercidin, ubenimex, zinostatin, zorubicin, denopterin, methotrexate, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, fluorouracil, tegafur, L-asparaginase, pulmozyme, aceglatone, aldophosphamide glycoside, aminolevulinic acid, amsacrine, bestrabucil, bisantrene, carboplatin, cisplatin, defofamide, demecolcine, diaziquone, elformithine, elliptinium acetate, etoglucid, etoposide, flutamide, gallium nitrate, hydroxyurea, interferon-alpha, interferon-beta, interferon-gamma, interleukin-2, lentinan, lonidamine, mitoguazone, mitoxantrone, mopidamol, nitracrine, pentostatin, phenamet, pirarubicin, podophyllinic acid, 2-ethylhydrazide, procarbazine, razoxane, sizofuran, spirogermanium, paclitaxel, tamoxifen, teniposide, tenuazonic acid, triaziquone, 2,2′,2″-trichlorotriethylamine, urethane, vinblastine, vincristine, and vindesine.

Pharmacology and Utility

Compounds of the invention are screened against the kinase panel (wild type and/or mutation thereof) and may modulate the activity of at least one kinase panel member. As such, compounds of the invention may be useful for treating diseases or disorders in which kinases contribute to the pathology and/or symptomology of the disease. Examples of kinases that may be inhibited by the compounds and compositions described herein and against which the methods described herein may be useful include, but are not limited to Alk, Abl, Aurora-A, B-Raf, C-Raf, Bcr-Abl, BRK, Blk, Bmx, BTK, C-Kit, C-Src, EphB1, EphB2, EphB4, FLT1, Fms, Flt3, Fyn, FRK3, JAK2, KDR, Lck, Lyn, PDGFRα, PDGFRβ, PKCα, p38 (p38 MAP kinase, SAPK2α), Src, SIK, Syk, Tie2 and TrkB kinases, and mutant forms thereof.

The Ras-Raf-MEK-ERK signaling pathway mediates cellular response to growth signals. Ras is mutated to an oncogenic form in approximately 15% of human cancer. The Raf family belongs to the serine/threonine protein kinase and it includes three members, A-Raf, B-Raf and C-Raf (or Raf-1). The focus on Raf being a drug target has centered on the relationship of Raf as a downstream effector of Ras. However, B-Raf may have a prominent role in the formation of certain tumors with no requirement for an activated Ras allele (Nature 417:949-954 (2002). In particular, B-Raf mutations have been detected in a large percentage of malignant melanomas. Existing medical treatments for melanoma are limited in their effectiveness, especially for late stage melanomas. The compounds of the present invention also inhibit cellular processes involving B-Raf kinase, providing a new therapeutic opportunity for treatment of human cancers, such as melanoma.

Certain abnormal proliferative conditions are believed to be associated with Raf expression and are, therefore, believed to be responsive to inhibition of Raf expression. Abnormally high levels of expression of the Raf protein are also implicated in abnormal cell proliferation. These abnormal proliferative conditions are also believed to be responsive to inhibition of Raf kinase expression. For example, expression of the C-Raf protein is believed to play a role in abnormal cell proliferation since it has been reported that 60% of all lung carcinoma cell lines express unusually high levels of C-Raf mRNA and protein. Further examples of abnormal proliferative conditions are hyper-proliferative disorders such as cancers, hyperplasia, pulmonary fibrosis, angiogenesis, psoriasis, atherosclerosis and smooth muscle cell proliferation in the blood vessels, such as stenosis or restenosis following angioplasty. The cellular signaling pathway of which Raf is a part has also been implicated in inflammatory disorders characterized by T-cell proliferation (T-cell activation and growth), such as tissue graft rejection, endotoxin shock, and glomerular nephritis, for example.

The compounds of the present invention may also inhibit cellular processes involving C-Raf kinase. C-Raf is activated by the Ras oncogene, which is mutated in a wide number of human cancers. Therefore inhibition of the kinase activity of C-Raf may provide a way to prevent Ras mediated tumor growth [Campbell, S. L., Oncogene, 17, 1395 (1998)].

The Src family of kinases is implicated in cancer, immune system dysfunction and bone remodeling diseases. Members of the Src family include the following eight kinases in mammals: Src, Fyn, Yes, Fgr, Lyn, Hck, Lck, and Blk. For general reviews, see Thomas and Brugge, Annu. Rev. Cell Dev. Biol. (1997) 13, 513; Lawrence and Niu, Pharmacol. Ther. (1998) 77, 81; Tatosyan and Mizenina, Biochemistry (Moscow) (2000) 65, 49; Boschelli et al., Drugs of the Future 2000, 25(7), 717.

Fyn encodes a membrane-associated tyrosine kinase that has been implicated in the control of cell growth.

Lck plays a role in T-cell signaling. Mice that lack the Lck gene have a poor ability to develop thymocytes. The function of Lck as a positive activator of T-cell signaling suggests that Lck inhibitors may be useful for treating autoimmune disease such as rheumatoid arthritis. Molina et al., Nature, 357, 161 (1992). Hck, Fgr and Lyn have been identified as important mediators of integrin signaling in myeloid leukocytes. Lowell et al., J. Leukoc. Diol., 65, 313 (1999). Inhibition of these kinase mediators may therefore be useful for treating inflammation. Boschelli et al., Drugs of the Future, 2000, 25(7), 717.

Lyn, a member of the Src family, plays a role in the regulation of B-cell immune responses. Lyn-deficient mice display disrupted B-cell function, leading to autoimmunity and defective mast cell degranulation. Studies have also suggested that Lyn is a negative regulator of apoptosis in various cell systems. In leukemic cells, Lyn is constitutively activated, and the inhibition of Lyn expression reversed proliferation. In addition, Lyn has been shown to be expressed in colon and PC cells, and that overexpression of a dominant active Lyn in colon cancer cell lines induced chemoresistance. (Goldenberg-Furmanov et al., Cancer Res. 64:1058-1066 (2004)).

The kinase, c-Src transmits oncogenic signals of many receptors. For example, over-expression of EGFR or HER2/neu in tumors leads to the constitutive activation of C-Src, which is characteristic for the malignant cell but absent from the normal cell. On the other hand, mice deficient in the expression of C-Src exhibit an osteopetrotic phenotype, indicating a key participation of C-Src in osteoclast function and a possible involvement in related disorders. C-Src tyrosine kinase (CSK) influences the metastatic potential of cancer cells, particularly colon cancer.

C-Kit has a substantial homology to the PDGF receptor and to the CSF-1 receptor (c-Fms). Investigations on various erythroid and myeloid cell lines indicate an expression of the C-Kit gene in early stages of differentiation (Andre et al., Oncogene 4 (1989), 1047-1049). Certain tumors such as glioblastoma cells likewise exhibit a pronounced expression of the C-Kit gene.

Eph receptors, which include EphA and EphB subfamily, consist of the largest group of receptor tyrosine kinases. EphB was found to be overexpressed in several tumors including ovarian tumors, liver tumors, kidney tumors as well as melanomas. Downregulation of EphB signaling has shown to inhibit tumor growth and metastasis. Therefore, EphB may be an important target for anti-tumorigenic therapies. (Clevers et al., Cancer Res. 66:2-5 (2006); Heroult et al., Experimental Cell Res. 312: 642-650 (2006); and Batlle et al., Nature 435:1126-1130 (2005)).

Kinase insert domain-containing receptor (referred to as “KDR” hereinafter) [WO 92/14748; Proc. Natl. Acad. Sci. USA, 88: 9026 (1991)]; Biochem. Biophys. Res. Comm., 187: 1579 (1992); WO 94/11499) and Fms-like tyrosine kinase (referred to as “Flt1” hereinafter) [Oncogene, 5: 519 (1990); Science, 255: 989 (1992)] belong to the receptor type tyrosine kinase family. It has been reported that VEGF specifically binds to Flt-1 and KDR at Kd values of 20 pM and 75 pM and that Flt1 and KDR are expressed in vascular endothelial cells in a specific manner [Proc. Natl. Acad. Sci. USA, 90: 7533 (1993); Proc. Natl. Acad. Sci. USA, 90: 8915 (1993)]. With regard to Flt-1 in various diseases, it has been reported that, in comparison with vascular endothelial cells in normal tissues, expression of Flt-1 mRNA increases in tumor vascular endothelial cells of human glioblastoma tissues [Nature, 359: 845 (1992)] and tumor vascular endothelial cells of human digestive organ cancer tissues [Cancer Research, 53: 4727 (1993)]. Additionally, it has been reported that expression of Flt-1 mRNA is observed by in situ hybridization in vascular endothelial cells of joints of patients with rheumatoid arthritis [J. Experimental Medicine, 180: 341 (1994)]. Studies also suggest that Flt-1 plays an important role in tumor angiogenesis.

Flt3 is a member of the type III receptor tyrosine kinase (RTK) family. Flt3 (Fms-like tyrosine kinase) is also known as Flk-2 (fetal liver kinase 2). Aberrant expression of the Flt3 gene has been documented in both adult and childhood leukemias including acute myeloid leukemia (AML), AML with trilineage myelodysplasia (AML/TMDS), acute lymphoblastic leukemia (ALL), and myelodysplastic syndrome (MDS). In approximately 25% of AML, the leukemia cells express a constitutively active form of auto-phosphorylated (p) FLT3 tyrosine kinase on the cell surface. The activity of p-FLT3 confers growth and survival advantage on the leukemic cells Inhibition of p-FLT3 kinase activity induces apoptosis (programmed cell death) of the leukemic cells.

Abelson tyrosine kinase (i.e. Abl, c-Abl) is involved in the regulation of the cell cycle, in the cellular response to genotoxic stress, and in the transmission of information about the cellular environment through integrin signaling. The Abl protein appears to serve a complex role as a cellular module that integrates signals from various extracellular and intracellular sources and that influences decisions in regard to cell cycle and apoptosis. Abelson tyrosine kinase includes sub-types derivatives such as the chimeric fusion (oncoprotein) Bcr-Abl with deregulated tyrosine kinase activity or the v-Abl. Bcr-Abl is important in the pathogenesis of 95% of chronic myelogenous leukemia (CML) and 10% of acute lymphocytic leukemia.

Compounds of the present invention may inhibit Abl kinase, for example, v-Abl kinase. The compounds of the present invention may also inhibit wild-type Bcr-Abl kinase and mutations of Bcr-Abl kinase, and thus may be suitable for the treatment of Bcr-Abl-positive cancer and tumor diseases, such as leukemias (e.g., chronic myeloid leukemia and acute lymphoblastic leukemia) and other proliferation disorders related to Bcr-Abl. Compounds of the present invention may also be effective against leukemic stem cells, and may be potentially useful for the purification of these cells in vitro after removal of said cells (for example, bone marrow removal), and reimplantation of the cells once they have been cleared of cancer cells (for example, reimplantation of purified bone marrow cells).

Anaplastic lymphoma kinase (ALK), a member of the insulin receptor superfamily of receptor tyrosine kinases, has been implicated in oncogenesis in hematopoietic and non-hematopoietic tumors. The aberrant expression of full-length ALK receptor proteins has been reported in neuroblastomas and glioblastomas; and ALK fusion proteins have occurred in anaplastic large cell lymphoma. The study of ALK fusion proteins has also raised the possibility of new therapeutic treatments for patients with ALK-positive malignancies. (Pulford et al., Cell. Mol. Life Sci. 61:2939-2953 (2004)).

Aurora-A, a serine/threonine mitotic kinase, has been reported to be overexpressed in various human cancers, and its overexpression induces aneuploidy, centrosome amplification and tumorigenic transformation in cultured human and rodent cells. (Zhang et al., Oncogene 23:8720-30 (2004)).

Bmx/Etk non-receptor tyrosine protein kinase has been implicated in endothelial cell migration and tube formation in vitro. Bmx in endothelium and bone marrow has also been reported to play an important role in arteriogenesis and angiogenesis in vivo, suggesting that Bmx may be a novel target for the treatment of vascular diseases such as coronary artery disease and peripheral arterial disease. (He et al., J. Clin. Invest. 116:2344-2355 (2006)).

Bruton's tyrosine kinase (BTK) gene encodes a cytoplasmic tyrosine kinase that plays an essential role in mediating BCR signaling. (de Weers et al., J. Biol. Chem. 269:23857-23860 (1994); Kurosaki et al., Immunity. 12:1-5 (2000)). Defects in the BTK gene cause Agammaglobulinemia, an X-linked immunodeficiency characterized by failure to produce mature B lymphocyte cells and associated with a failure of Ig heavy chain rearrangement.

Breast tumor kinase (Brk) is a soluble protein-tyrosine kinase overexpressed in the majority of breast cancers and also in normal skin and gut epithelium, but not in normal breast epithelial cells. (Zhang et al., J Biol. Chem. 280:1982-1991 (2005)).

The Janus kinases (JAK) are a family of tyrosine kinases consisting of JAK1, JAK2, JAK3 and TYK2. The JAKs play an important role in cytokine signaling. The down-stream substrates of the JAK family of kinases include the signal transducer and activator of transcription (STAT) proteins. JAK/STAT signaling has been implicated in the mediation of many abnormal immune responses such as allergies, asthma, autoimmune diseases such as transplant rejection, rheumatoid arthritis, amyotrophic lateral sclerosis and multiple sclerosis, as well as in solid and hematologic malignancies such as leukemias and lymphomas.

An important factor in the tumor angiogenesis is vascular endothelium growth factor (VEGF). VEGF can promote and maintain the establishment of tumor vascular system, and can also promote the tumor growth directly. VEGF can induce the mitogenesis and chemotaxis of vascular endothelial cell (VEC) and tumor cell (TC). Almost all types of TC can secret VEGF, but the expression of VEGF in the normal tissue is very low. In the four VEGF receptors, KDR is the main receptor which gives play to VEGF functions. KDR is highly expressed on tumor VEC while lowly expressed on the normal tissues. (Ren et al., World J. Gastroentrol. 8:596-601 (2002)).

Mitogen-activated protein kinases (MAPKs) are members of conserved signal transduction pathways that activate transcription factors, translation factors and other target molecules in response to a variety of extracellular signals. MAPKs are activated by phosphorylation at a dual phosphorylation motif having the sequence Thr-X-Tyr by mitogen-activated protein kinase kinases (MKKs). In higher eukaryotes, the physiological role of MAPK signaling has been correlated with cellular events such as proliferation, oncogenesis, development and differentiation. Accordingly, the ability to regulate signal transduction via these pathways (particularly via MKK4 and MKK6) could lead to the development of treatments and preventive therapies for human diseases associated with MAPK signaling, such as inflammatory diseases, autoimmune diseases and cancer.

Multiple forms of p38 MAPK (α, β, γ, δ), each encoded by a separate gene, form part of a kinase cascade involved in the response of cells to a variety of stimuli, including osmotic stress, UV light and cytokine mediated events. These four isoforms of p38 are thought to regulate different aspects of intracellular signaling. Its activation is part of a cascade of signaling events that lead to the synthesis and production of pro-inflammatory cytokines like TNFα. P38 functions by phosphorylating downstream substrates that include other kinases and transcription factors. Agents that inhibit p38 kinase have been shown to block the production of cytokines, including but not limited to TNFα, IL-6, IL-8 and IL-1β. Peripheral blood monocytes (PBMCs) have been shown to express and secrete pro-inflammatory cytokines when stimulated with lipopolysaccharide (LPS) in vitro. P38 inhibitors efficiently block this effect when PBMCs are pretreated with such compounds prior to stimulation with LPS. P38 inhibitors are efficacious in animal models of inflammatory disease. The destructive effects of many disease states are caused by the over production of pro-inflammatory cytokines. The ability of p38 inhibitors to regulate this overproduction makes them useful as disease modifying agents.

Molecules that block p38's function have been shown to be effective in inhibiting bone resorption, inflammation, and other immune and inflammation-based pathologies. Therefore, compounds of the invention that inhibit p38 activity are useful for the treatment of inflammation, osteoarthritis, rheumatoid arthritis, cancer, autoimmune diseases, and for the treatment of other cytokine mediated diseases.

PDGF (Platelet-derived Growth Factor) is a commonly occurring growth factor, which plays an important role both in normal cell growth and pathological cell proliferation, as seen in carcinogenesis and in diseases of the smooth-muscle cells of blood vessels, for example in atherosclerosis and thrombosis. Compounds of the invention may inhibit PDGF receptor (PDGFR) activity, and may therefore be suitable for the treatment of tumor diseases, such as gliomas, sarcomas, prostate tumors, and tumors of the colon, breast, and ovary.

Compounds of the present invention, may be used not only for treating tumors, for example in small cell lung cancer, but also as an agent to treat non-malignant proliferative disorders, such as atherosclerosis, thrombosis, psoriasis, scleroderma and fibrosis. Compounds of the present invention may also be useful for the protection of stem cells, for example to combat the hemotoxic effect of chemotherapeutic agents, such as 5-fluoruracil, and in asthma. Compounds of the invention may especially be used for the treatment of diseases due to the overexpression of PDGF receptor kinase.

Compounds of the present invention may exhibit useful effects in the treatment of disorders arising as a result of transplantation, for example, allogenic transplantation, especially tissue rejection, such as obliterative bronchiolitis (OB), i.e. a chronic rejection of allogenic lung transplants. In contrast to patients without OB, those with OB often show an elevated PDGF level in bronchioalveolar lavage fluids.

Compounds of the present invention may also be effective against diseases associated with vascular smooth-muscle cell migration and proliferation (where PDGF and PDGFR often also play a role), such as restenosis and atherosclerosis. These effects and the consequences thereof for the proliferation or migration of vascular smooth-muscle cells in vitro and in vivo may be demonstrated by administration of the compounds of the present invention, and also by investigating its effect on the thickening of the vascular intima following mechanical injury in vivo.

Protein kinase C (PKC) functions in processes relevant to carcinogenesis, tumor cell metastasis, and apoptosis. PKCα is associated with a diverse range of cancers, and is previously shown to be overexpressed in three out of four antiestrogen resistant breast cancer cell lines. (Frankel et al., Breast Cancer Res Treat. 2006 Oct. 24 (ePub)).

The stress activated protein kinases (SAPKs) are a family of protein kinases that represent the penultimate step in signal transduction pathways that result in activation of the c-Jun transcription factor and expression of genes regulated by c-Jun. In particular, c-Jun is involved in the transcription of genes that encode proteins involved in the repair of DNA that is damaged due to genotoxic insults. Therefore, agents that inhibit SAPK activity in a cell prevent DNA repair and sensitize the cell to agents that induce DNA damage or inhibit DNA synthesis and induce apoptosis of a cell or that inhibit cell proliferation.

The region encompassing the SNF1LK locus (also known as SIK) has been implicated in congenital heart defects often observed in patients with Down syndrome. Snf1lk is also expressed in skeletal muscle progenitor cells of the somite beginning at 9.5 dpc, suggesting a more general role for SNF1LK in the earliest stages of muscle growth and/or differentiation. (Genomics 83:1105-15 (2004)).

Syk is a tyrosine kinase that plays an important role in mast cell degranulation and eosinophil activation. Accordingly, Syk kinase is implicated in various allergic disorders, in particular asthma. It has been shown that Syk binds to the phosphorylated gamma chain of the FcεR1 receptor via N-terminal SH₂ domains, and is important for downstream signaling.

An inhibition of tumor growth and vascularization, and a decrease in lung metastases during adenoviral infections or during injections of the extracellular domain of Tie-2 (Tek) have been shown in breast tumor and melanoma xenograft models. (Lin et al., J. Clin. Invest. 100, 8: 2072-2078 (1997) and P. Lin, PNAS 95, 8829-8834, (1998)). Tie2 inhibitors can be used in situations where neovascularization takes place inappropriately (i.e. in diabetic retinopathy, chronic inflammation, psoriasis, Kaposi's sarcoma, chronic neovascularization due to macular degeneration, rheumatoid arthritis, infantile haemangioma and cancers).

The Trk family of neurotrophin receptors (TrkA, TrkB, TrkC) promotes the survival, growth and differentiation of the neuronal and non-neuronal tissues. The TrkB protein is expressed in neuroendocrine-type cells in the small intestine and colon, in the alpha cells of the pancreas, in the monocytes and macrophages of the lymph nodes and of the spleen, and in the granular layers of the epidermis. Expression of the TrkB protein has been associated with an unfavorable progression of Wilms tumors and of neuroblastomas. Moreover, TrkB is expressed in cancerous prostate cells but not in normal cells. The signaling pathway downstream of the Trk receptors involves the cascade of MAPK activation through the Shc, activated Ras, ERK-1 and ERK-2 genes, and the PLC-gamma transduction pathway (Sugimoto et al., Jpn J. Cancer Res. 2001 February; 92(2):152-60).

The class III receptor tyrosine kinases (RTKs), which include C-Fms, C-Kit, FLT3, platelet-derived growth factor receptor α (PDGFRα) and β (PDGFRβ), have been reported to be associated with the pathogenesis of an increasing number of malignancies. (Blume-Jensen et al., Nature 411:355-565 (2001); Scheijin et al., Oncogene 21:3314-3333 (2002)).

In accordance with the foregoing, the present invention further provides a method for preventing or treating any of the diseases or disorders described above in a subject in need of such treatment, which method comprises administering to said subject a therapeutically effective amount of a compound of Formula (1), (2) or (3), or a pharmaceutically acceptable salt thereof. For any of the above uses, the required dosage will vary depending on the mode of administration, the particular condition to be treated and the effect desired. (See, “Administration and Pharmaceutical Compositions,” infra)

Administration and Pharmaceutical Compositions

In general, compounds of the invention will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. In general, satisfactory results are indicated to be obtained systemically at daily dosages of from about 0.03 to 2.5 mg/kg per body weight. An indicated daily dosage in the larger mammal, e.g. humans, is in the range from about 0.5 mg to about 100 mg, conveniently administered, e.g. in divided doses up to four times a day or in retard form. Suitable unit dosage forms for oral administration comprise from ca. 1 to 50 mg active ingredient.

Compounds of the invention may be administered as pharmaceutical compositions by any conventional route, in particular enterally, e.g., orally in the form of tablets or capsules; parenterally, e.g., in the form of injectable solutions or suspensions; topically, e.g., in the form of lotions, gels, ointments or creams; or in a nasal or suppository form.

Pharmaceutical compositions comprising a compound of the present invention in free form or in a pharmaceutically acceptable salt form in association with at least one pharmaceutically acceptable carrier or diluent may be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; and/or b) lubricants, e.g., silica, talcum, stearic acid or its magnesium or calcium salt and/or polyethylene glycol. Tablets may further comprise c) binders, e.g., magnesium aluminum silicate, starch paste, gelatin, tragacanth, methylcellulose, sodium carboxymethylcellulose and or polyvinylpyrrolidone; and if desired, d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions.

The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Suitable formulations for transdermal applications include an effective amount of a compound of the present invention with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used. Suitable formulations for topical application, e.g., to the skin and eyes, may be aqueous solutions, ointments, creams or gels well-known in the art. Such may contain solubilizers, stabilizers, tonicity enhancing agents, buffers and preservatives.

Compounds of the invention may be administered in therapeutically effective amounts in combination with one or more therapeutic agents (pharmaceutical combinations). For example, synergistic effects can occur with other immunomodulatory or anti-inflammatory substances, for example when used in combination with cyclosporin, rapamycin, or ascormycin, or immunosuppressant analogues thereof, for example cyclosporin A (CsA), cyclosporin G, FK-506, rapamycin, or comparable compounds, corticosteroids, cyclophosphamide, azathioprine, methotrexate, brequinar, leflunomide, mizoribine, mycophenolic acid, mycophenolate mofetil, 15-deoxyspergualin, immunosuppressant antibodies, especially monoclonal antibodies for leukocyte receptors, for example MHC, CD2, CD3, CD4, CD7, CD25, CD28, B7, CD45, CD58 or their ligands, or other immunomodulatory compounds, such as CTLA41g. Where the compounds of the invention are administered in conjunction with other therapies, dosages of the co-administered compounds will of course vary depending on the type of co-drug employed, on the specific drug employed, on the condition being treated and so forth.

The invention also provides for a pharmaceutical combinations, e.g. a kit, comprising a) a first agent which is a compound of the invention as disclosed herein, in free form or in pharmaceutically acceptable salt form, and b) at least one co-agent. The kit can comprise instructions for its administration.

Processes for Making Compounds of the Invention

General procedures for preparing compounds of the invention are described in the Examples, infra. In the reactions described, reactive functional groups, for example hydroxy, amino, imino, thio or carboxy groups, where these are desired in the final product, may be protected to avoid their unwanted participation in the reactions. Conventional protecting groups may be used in accordance with standard practice (see e.g., T. W. Greene and P. G. M. Wuts in “Protective Groups in Organic Chemistry”, John Wiley and Sons, 1991).

A compound of the invention may be prepared as a pharmaceutically acceptable acid addition salt by reacting the free base form of the compound with a pharmaceutically acceptable inorganic or organic acid. Alternatively, a pharmaceutically acceptable base addition salt of a compound of the invention may be prepared by reacting the free acid form of the compound with a pharmaceutically acceptable inorganic or organic base. Alternatively, the salt forms of the compounds of the invention may be prepared using salts of the starting materials or intermediates.

The free acid or free base forms of the compounds of the invention may be prepared from the corresponding base addition salt or acid addition salt from, respectively. For example, a compound of the invention in an acid addition salt form may be converted to the corresponding free base by treating with a suitable base (e.g., ammonium hydroxide solution, sodium hydroxide, and the like). A compound of the invention in a base addition salt form may be converted to the corresponding free acid by treating with a suitable acid (e.g., hydrochloric acid, etc.).

Compounds of the invention in unoxidized form may be prepared from N-oxides of compounds of the invention by treating with a reducing agent (e.g., sulfur, sulfur dioxide, triphenyl phosphine, lithium borohydride, sodium borohydride, phosphorus trichloride, tribromide, or the like) in a suitable inert organic solvent (e.g. acetonitrile, ethanol, aqueous dioxane, or the like) at 0 to 80° C.

Prodrug derivatives of the compounds of the invention may be prepared by methods known to those of ordinary skill in the art (See e.g., Saulnier et al., (1994), Bioorganic and Medicinal Chemistry Letters, Vol. 4, p. 1985). For example, appropriate prodrugs may be prepared by reacting a non-derivatized compound of the invention with a suitable carbamylating agent (e.g., 1,1-acyloxyalkylcarbanochloridate, para-nitrophenyl carbonate, or the like).

Compounds of the present invention may be conveniently prepared or formed during the process of the invention, as solvates (e.g., hydrates). Hydrates of compounds of the present invention may be conveniently prepared by recrystallization from an aqueous/organic solvent mixture, using organic solvents such as dioxin, tetrahydrofuran or methanol.

Compounds of the invention may be prepared as their individual stereoisomers by reacting a racemic mixture of the compound with an optically active resolving agent to form a pair of diastereoisomeric compounds, separating the diastereomers and recovering the optically pure enantiomers. Resolution of enantiomers may be carried out using covalent diastereomeric derivatives of the compounds of the invention, or by using dissociable complexes (e.g., crystalline diastereomeric salts). Diastereomers have distinct physical properties (e.g., melting points, boiling points, solubility, reactivity, etc.), and may be readily separated by taking advantage of these dissimilarities. The diastereomers may be separated by chromatography, or by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques, Andre Collet, Samuel H. Wilen, “Enantiomers, Racemates and Resolutions”, John Wiley And Sons, Inc., 1981.

In summary, compounds having Formula (1), (2) or (3) may be made by a process as described in the Examples; and

(a) optionally converting a compound of the invention into a pharmaceutically acceptable salt;

(b) optionally converting a salt form of a compound of the invention to a non-salt form;

(c) optionally converting an unoxidized form of a compound of the invention into a pharmaceutically acceptable N-oxide;

(d) optionally converting an N-oxide form of a compound of the invention to its unoxidized form;

(e) optionally resolving an individual isomer of a compound of the invention from a mixture of isomers;

(f) optionally converting a non-derivatized compound of the invention into a pharmaceutically acceptable prodrug derivative; and

(g) optionally converting a prodrug derivative of a compound of the invention to its non-derivatized form.

The present invention also includes all suitable isotopic variations of the compounds of the invention, or pharmaceutically acceptable salts thereof. An isotopic variation of a compound of the invention or a pharmaceutically acceptable salt thereof is defined as one in which at least one atom is replaced by an atom having the same atomic number but an atomic mass different from the atomic mass usually found in nature. Examples of isotopes that may be incorporated into the compounds of the invention and pharmaceutically acceptable salts thereof include but are not limited to isotopes of hydrogen, carbon, nitrogen and oxygen such as ²H, ³H, ¹¹C, ¹³C, ¹⁴C, ¹⁵N, ¹⁷O, ¹⁸O, ³⁵S, ¹⁸F, ³⁶Cl and ¹²³I. Certain isotopic variations of the compounds of the invention and pharmaceutically acceptable salts thereof, for example, those in which a radioactive isotope such as ³H or ¹⁴C is incorporated, are useful in drug and/or substrate tissue distribution studies. In particular examples, ³H and ¹⁴C isotopes may be used for their ease of preparation and detectability. In other examples, substitution with isotopes such as ²H may afford certain therapeutic advantages resulting from greater metabolic stability, such as increased in vivo half-life or reduced dosage requirements. Isotopic variations of the compounds of the invention or pharmaceutically acceptable salts thereof can generally be prepared by conventional procedures using appropriate isotopic variations of suitable reagents. Isotopic variations of the compounds have the potential to change a compound's metabolic fate and/or create small changes in physical properties such as hydrophobicity, and the like. Isotopic variation have the potential to enhance efficacy and safety, enhance bioavailability and half-life, alter protein binding, change biodistribution, increase the proportion of active metabolites and/or decrease the formation of reactive or toxic metabolites.

Insofar as the production of the starting materials is not particularly described, the compounds are known or may be prepared analogously to methods known in the art or as disclosed in the Examples hereinafter. One of skill in the art will appreciate that the above transformations are only representative of methods for preparation of the compounds of the present invention, and that other well known methods can similarly be used.

The following examples are offered to illustrate but not to limit the invention.

### Example 1

### N-{4-Methyl-3-[2-(6-methylamino-pyrimidin-4-yl)-2H-[1,2,4]triazol-3-ylamino]-phenyl}-3-trifluoromethyl-benzamide (5)

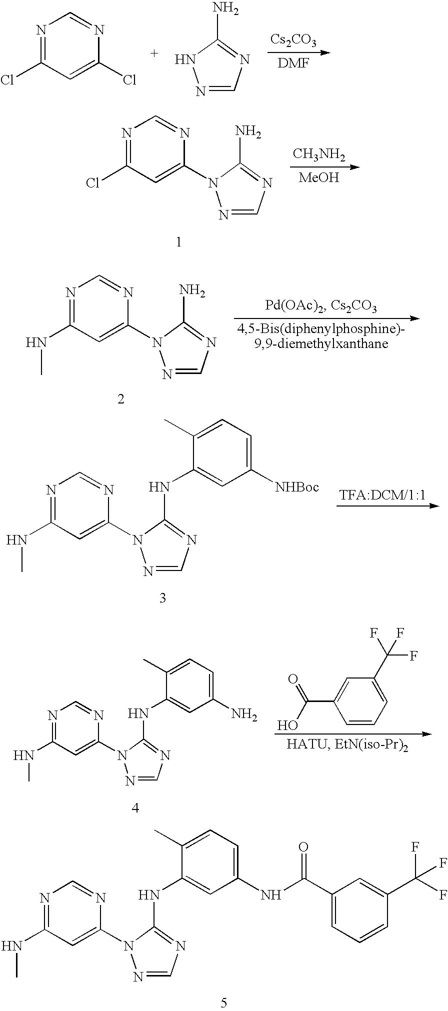

### 2-(6-Chloro-pyrimidin-4-yl)-2H-[1,2,4]triazol-3-ylamine (1)

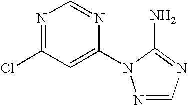

To a nitrogen purged flask is added 4,6-dichloropyrimidine (20 g, 0.134 mol), 3-amino-1,2,4-triazole (11.3 g, 0.134 mol), Cs₂CO₃ (43.7 g, 0.134 mol) and DMF (300 mL). After stirring at rt for 2 days, the mixture is diluted with EtOAc (800 mL), then washed with water (3×). The organic layer is dried with MgSO₄ and concentrated in vacuo. The residue is dissolved in hot CH₃CN (800 mL) and kept at rt overnight. The undesired isomer is precipitated out and removed by filtration. The filtrate is concentrated slowly at 70° C. until a yellow solid starts to precipitate out, then the solution is kept at rt overnight. The precipitate is collected by filtration, checked by TLC, and recrystallized as needed. ¹H NMR (400 MHz, DMSO) δ 8.90 (s, 1H), 7.90 (s, 2H), 7.80 (s, 1H), 7.79 (s, 1H). MS (ESI) m/z: 197 (M+H)⁺.

### [6-(5-Amino-[1,2,4]triazol-1-yl)-pyrimidin-4-yl]-methyl-amine (2)

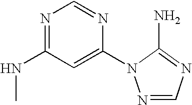

To a high pressure tube is added compound 1 (1.0 g), MeOH (20 mL) and CH₃NH₂ (20 mL, 1 M soln in MeOH). The mixture is heated to 50° C. for 2 hrs, then concentrated in vacuo, and the residue is purified by flash chromatography [silica gel, DCM:MeOH/9:1]. ¹H NMR (400 MHz, DMSO) δ 8.40 (s, 1H), 7.60 (s, 2H), 7.56 (s, 2H), 6.70 (s, 1H), 2.50 (s, 3H). MS (ESI) m/z: 192 (M+H)⁺.

### {4-Methyl-3-[2-(6-methylamino-pyrimidin-4-yl)-2H-[1,2,4]triazol-3-ylamino]-phenyl}-carbamic acid tert-butyl ester (3)

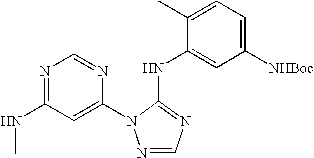

To a high pressure tube is added compound 2 (0.708 g, 3.71 mmol), (3-Bromo-4-methyl-phenyl)-carbamic acid tert-butyl ester (0.909 g, 4.08 mmol), Pd(OAc)₂ (83 mg, 0.37 mmol), Cs₂CO₃ (1.207 g, 3.70 mmol), 4,5-Bis(diphenylphosphine)-9,9-dimethylxanthane (0.214 g, 0.37 mmol) and 1,4-dioxane (10 mL). The mixture is flushed with N₂ at 0° C. for a few minutes, and heated to 90° C. overnight. The mixture is then poured into H₂O and extracted with EtOAc. The organic layer is separated, dried with MgSO₄, then concentrated in vacuo and the residue is purified by flash chromatography [silica gel, hexane:EtOAc/4:6]. MS (ESI) m/z: 397 (M+H)⁺.

### 4-Methyl-N3-[2-(6-methylamino-pyrimidin-4-yl)-2H-[1,2,4]triazol-3-yl]-benzene-1,3-diamine (4)

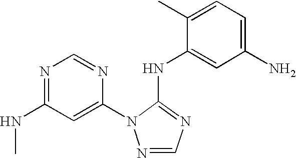

Compound 3 (0.80 g) is dissolved in a mixed solvent of DCM:TFA (10 mL:10 mL). The mixture is stirred at rt for 1 hr, then concentrated in vacuo. The residue is dissolved in EtOAc, then washed with aqueous NaHCO₃. The organic layer is dried with MgSO₄, then concentrated to give a yellow solid. MS (ESI) m/z: 297 (M+H)⁺.

### N-{4-Methyl-3-[2-(6-methylamino-pyrimidin-4-yl)-2H-[1,2,4]triazol-3-ylamino]-phenyl}-3-trifluoromethyl-benzamide (5)

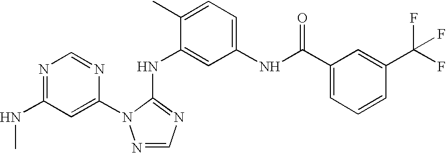

To a vial is added compound 4 (20 mg, 0.067 mmol), 3-trifluorobenzoic acid (13 mg, 0.067 mmol), HATU (26 mg, 0.067 mmol), EtN(iPr)₂ (11.7 μL, 0.122 mmol) and DMF (2 mL). The mixture is stirred at rt overnight, then purified by HPLC. ¹H NMR (400 MHz, DMSO) δ 11.20 (s, 1H), 10.50 (s, 1H), 8.70 (s, 1H), 8.55 (s, 1H), 8.30 (s, 1H), 8.28 (d, 1H), 7.95 (s, 1H), 7.90 (s, 1H), 7.80 (t, 1H), 7.45 (d, 1H), 7.26 (d, 1H), 6.85 (s, 1H), 2.90 (s, 3H), 2.40 (s, 3H). MS (ESI) m/z: 469 (M+H)⁺.

### Example 2

### 4-Methyl-3-[2-(6-methylamino-pyrimidin-4-yl)-2H-[1,2,4]triazol-3-ylamino]-N-(3-trifluoromethyl-phenyl)-benzamide (8)

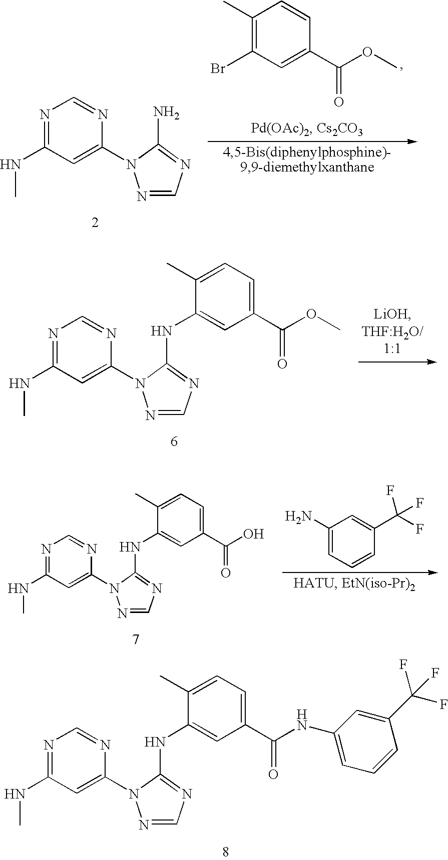

### 4-Methyl-3-[2-(6-methylamino-pyrimidin-4-yl)-2H-[1,2,4]triazol-3-ylamino]-benzoic acid methyl ester (6)

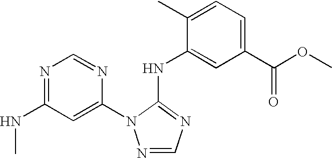

To a solution of 2 (1.82 g, 9.52 mmol) in dioxane (95 mL) is added Pd₂(dba)₃ (435 mg, 0.47 mmol), XantPhos (826 mg, 1.42 mmol), Cs₂CO₃ (7.44 g, 22.8 mmol) and 3-bromo-4-methyl-benzoic acid methyl ester (2.4 g, 10.5 mmol). The flask is purged with Argon, capped and heated at 90° C. overnight. The mixture is diluted with H₂O and extracted with ethyl acetate. The organic phase are washed with brine, dried over anhydrous Na₂SO₄, filtered and concentrated in vacuo. Flash column chromatography on silica gel (hexane:EtOAc/1:1) provided compound 6. MS (ESI) m/z: 340.1 (M+H)⁺.

### 4-Methyl-3-[2-(6-methylamino-pyrimidin-4-yl)-2H-[1,2,4]triazol-3-ylamino]-benzoic acid (7)

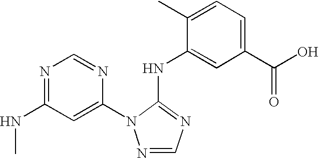

A mixture of compound 6 (800 mg, 2.36 mg) and LiOH.H₂O (990 mg, 2.6 mmol) in THF:H₂O/3:1 (24 mL) is stirred at rt overnight. The reaction mixture is concentrated in vacuo to remove most of THF. The mixture is cooled down to 0° C. and filtered to give the desired product 7 as a sodium salt. MS (ESI) m/z: 326.1 (M+H)⁺.

### 4-Methyl-3-[2-(6-methylamino-pyrimidin-4-yl)-2H-[1,2,4]triazol-3-ylamino]-N-(3-trifluoromethyl-phenyl)-benzamide (8)

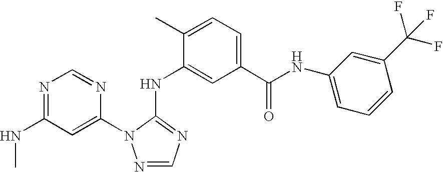

A mixture of 7 (27.8 mg, 0.08 mmol), 3-trifluoromethyl-phenylamine (21.4 mg, 0.098 mmol), HATU (42 mg, 0.11 mmol), and diisopropylethyl amine (41 μL, 0.24 mmol) in DMF (1.5 mL) is kept stirring for 15 hours. The reaction mixture is concentrated and purified by Prep-HPLC to afford 8. MS (ESI) m/z: 469.2 (M+1)⁺.

### Example 3

### 4-Methyl-3-(3-methyl-1-(6-(methylamino)pyrimidin-4-yl)-1H-1,2,4-triazol-5-ylamino)-N-(3-(4-methylpiperazin-1-yl)-5-(trifluoromethyl)phenyl)benzamide (11)

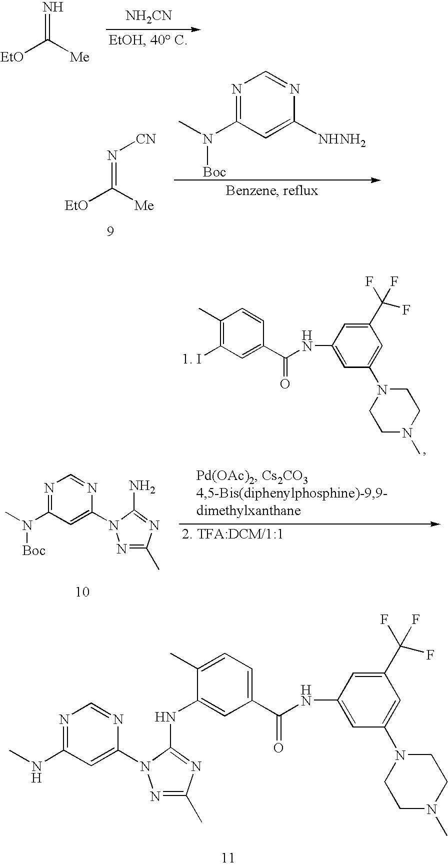

### (E)-ethyl N-cyanoacetimidate (9)

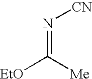

A solution of acetimidic acid ethyl ester (5.0 g, 40.4 mmol) and cyanamide (8.6 g, 205.1 mmol) in ethanol (60 mL) is heated at 40° C. under nitrogen for 2 hrs. The ammonium chloride formed during the reaction is filtered off, and the filtrate is concentrated in vacuo to afford crude product. The crude product is dissolved in ethyl acetate and washed with water and brine. The combined organic layers are dried with MgSO₄ and concentrated in vacuo to give (E)-ethyl N-cyanoacetimidate as a white solid. ¹H NMR (400 MHz, CD₃OD) δ 4.14 (q, J=7.0 Hz, 2H), 1.98 (s, 3H), 1.23 (t, J=7.0 Hz, 3H).

### 6-(5-Amino-3-methyl-[1,2,4]triazol-1-yl)-pyrimidin-4-yl]-methyl-carbamic acid tert-butyl ester (10)

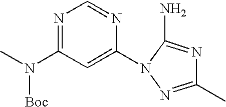

A mixture of (E)-ethyl N-cyanoacetimidate (1.2 g, 10.7 mmol) and tert-butyl 6-hydrazinylpyrimidin-4-yl (methyl)carbamate (1.7 g, 7.13 mmol) in toluene (12 mL) is refluxed overnight. The reaction mixture is purified on chromatography (silica gel, 10-50% EtOAc/Hexane) to give the desired product as a white solid. ¹H NMR (400 MHz, CD₃OD) δ 8.70 (d, J=0.8 Hz, 1H), 8.44 (d, J=0.8 Hz, 1H), 3.44 (s, 3H), 2.38 (s, 3H), 1.53 (s, 9H). MS (ESI) m/z: 306.1 (M+H)⁺.

### 4-Methyl-3-(3-methyl-1-(6-(methylamino)pyrimidin-4-yl)-1H-1,2,4-triazol-5-ylamino)-N-(3-(4-methylpiperazin-1-yl)-5-(trifluoromethyl)phenyl)benzamide (11)

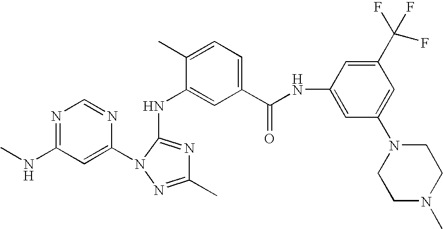

A mixture of compound 10 (46.3 mg, 0.15 mmol), 3-iodo-4-methyl-N-(3-(4-methylpiperazin-1-yl)-5-(trifluoromethyl)phenyl)benzamide (84.0 mg, 0.167 mmol), Pd₂(dba)₃ (5.0 mg, 0.005 mmol), Xantphos (16.7 mg, 0.029 mmol) and Cs₂CO₃ (100 mg, 0.31 mmol) in dioxane (4.0 mL) is heated to 150° C. for 20 min under microwave condition. The resulting reaction mixture is purified by HPLC to afford product. ¹H NMR (400 MHz, CD₃OD) δ 8.49 (s, 1H), 8.34 (s, 1H), 7.76 (s, 1H), 7.48-7.46 (m, 2H), 7.33 (d, J=8.0 Hz, 1H), 6.99 (s, 1H), 6.69 (s, 1H), 3.50-3.28 (m, 8H), 2.88 (s, 3H), 2.86 (s, 3H), 2.40 (s, 3H), 2.26 (s, 3H). MS (ESI) m/z: 581.2 (M+H)⁺.

### Example 4

### 1-{4-Methyl-3-[2-(6-methylamino-pyrimidin-4-yl)-2H-[1,2,4]triazol-3-ylamino]-phenyl}-3-(3-pyrrolidin-1-ylmethyl-5-trifluoromethyl-phenyl)-urea (12)

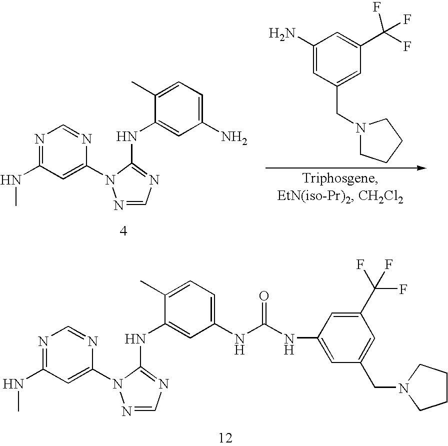

A solution of 4 (29.6 mg, 0.1 mmol) and diisopropylethyl amine (38 μL, 0.22 mmol) in 2 mL CH₂Cl₂ is added dropwise to a CH₂Cl₂ (1 mL) solution of triphosgene (11 mg, 0.37 mmol) under N₂. The mixture is stirred at room temperature for 15 min. This solution is added dropwise to a solution of 3-pyrrolidin-1-ylmethyl-5-trifluoromethyl-phenylamine (27 mg, 0.11 mmol) and diisopropanylethylamine (32 μL, 0.18 mmol) in CH₂Cl₂ (2 mL) over 2 min. The mixture is stirred at rt for 30 min. The solvent is removed in vacuo and the crude product is purified by HPLC to afford compound 12. LC-MS (ESI) m/z: 567.2 (M+H)⁺.

Table 1 describes representative compounds obtained by the above Examples.

TABLE 1

| Com- | | Physical data (¹H NMR (400 MHz, |
|---|---|---|
| pound | Structure | DMSO), MS (ESI) m/z) |
| 13 | 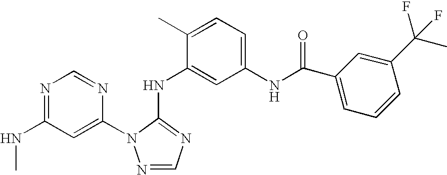 | ¹H NMR (400 MHz, DMSO) δ 8.75 (s, 1H), 8.55 (s, 1H), 8.50 (s, 1H), 8.15 (s, 1H), 8.10 (d, 1H), 7.95 (s, 1H), 7.90 (s, 1H), 7.80 (d, 1H), 7.66 (t, 1H), 7.45 (d, 1H), 7.20 (d, 1H), 6.85 (s, 1H), 2.90 (s, 3H), 2.50 (s, 3H), 2.05 (t, 3H). MS (ESI) m/z: 465.1 (M + H)⁺. |
| 14 | 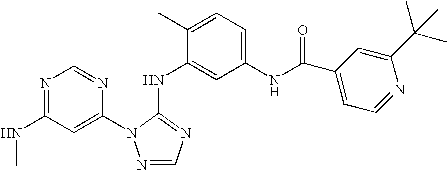 | ¹H NMR (400 MHz, DMSO) δ 8.75 (m, 2H), 8.70 (s, 1H), 7.94 (s, 1H), 7.90 (s, 1H), 7.75 (d, 1H), 7.40 (d, 1H), 7.25 (d, 1H), 6.85 (s, 1H), 2.95 (s, 3H), 2.35 (s, 3H), 1.40 (s, 6H). MS (ESI) m/z: 458.20 (M + H)⁺ |
| 15 | 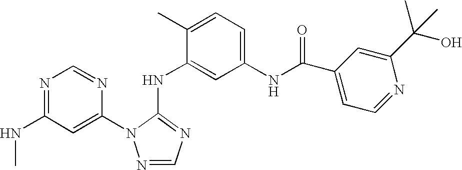 | ¹H NMR (400 MHz, DMSO) δ 8.70 (m, 2H), 8.65 (s, 1H), 8.25 (s, 1H), 8.00 (s, 1H), 7.95 (s, 1H), 7.80 (s, 1H), 7.45 (d, 1H), 7.25 (d, 1H), 6.85 (s, 1H), 2.90 (s, 3H), 2.38 (s, 3H), 1.50 (s, 3H). MS (ESI) m/z: 460.1 (M + H)⁺ |
| 16 | 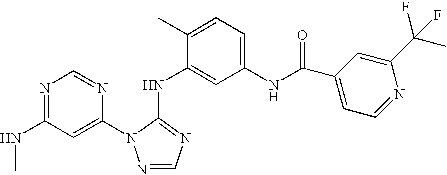 | ¹H NMR (400 MHz, DMSO) δ 8.88 (d, 1H), 8.75 (s, 1H), 8.55 (s, 1H), 8.20 (s, 1H), 8.05 (d, 1H), 7.95 (s, 1H), 7.90 (s, 1H), 7.45 (d, 1H), 7.25 (d, 1H), 6.85 (s, 1H), 2.92 (s, 3H), 2.45 (s, 3H), 2.05 (t, 3H). MS (ESI) m/z: 466.10 (M + H)⁺ |
| 17 | 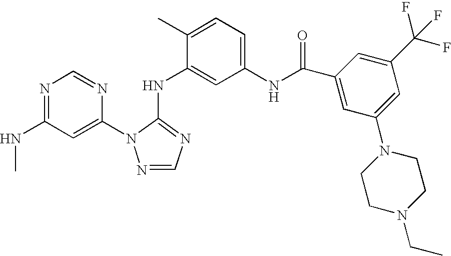 | ¹H NMR (400 MHz, DMSO) δ 9.65 (s, 1H), 8.70 (s, 1H), 8.65 (s, 1H), 7.95 (s, 1H), 7.90 (s, 1H), 7.80 (s, 1H), 7.78 (s, 1H), 7.50 (s, 1H), 7.40 (d, 1H), 7.20 (d, 1H), 6.85 (s, 1H), 4.10 (m, 2H), 3.60 (m, 2H), 3.25 (m, 2H), 3.12 (m, 4H), 2.92 (s, 3H), 2.35 (s, 3H), 1.25 (t, 3H). MS (ESI) m/z: 581.2 (M + H)⁺ |
| 18 | 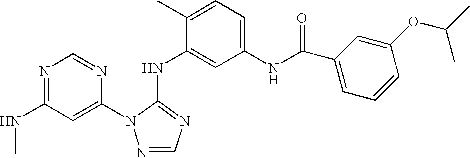 | ¹H NMR (400 MHz, DMSO) δ 8.72 (s, 1H), 8.55 (s, 1H), 7.95 (s, 1H), 7.90 (s, 1H), 7.50 (m, 2H), 7.40 (m, 2H), 7.20 (d, 1H), 7.10 (d, 1H), 6.85 (s, 1H), 4.70 (m, 1H), 2.90 (s, 3H), 2.35 (s, 3H), 1.30 (d, 6H). MS (ESI) m/z: 459.2 (M + H)⁺ |
| 19 | 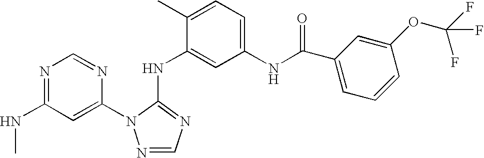 | ¹H NMR (400 MHz, DMSO) δ 8.75 (s, 1H), 8.55 (s, 1H), 8.00 (d, 1H), 7.90 (s, 1H), 7.85 (s, 1H), 7.70 (t, 2H), 7.60 (m, 1H), 7.40 (d, 1H), 7.25 (d, 1H), 6.85 (s, 1H), 6.65 (s, 1H), 2.88 (s, 3H), 2.35 (s, 3H). MS (ESI) m/z: 485.20 (M + H)⁺ |
| 20 | 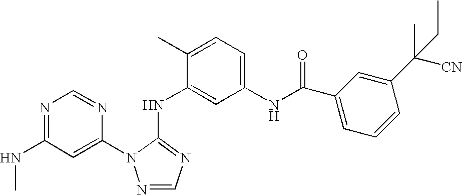 | ¹H NMR (400 MHz, DMSO) δ 8.72 (s, 1H), 8.55 (s, 1H), 8.00 (s, 1H), 7.95 (d, 1H), 7.90 (s, 1H), 7.70 (d, 1H), 7.60 (t, 1H), 7.46 (m, 1H), 7.40 (m, 1H), 7.22 (d, 1H), 6.85 (s, 1H), 2.90 (s, 3H), 2.38 (s, 3H), 2.05 (m, 2H), 1.70 (s, 3H), 0.86 (t, 3H). MS (ESI) m/z: 482.2 (M + H)⁺ |
| 21 | 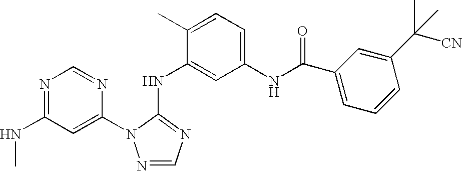 | ¹H NMR (400 MHz, DMSO) δ 8.72 (s, 1H), 8.55 (s, 1H), 8.05 (s, 1H), 7.95 (d, 1H), 7.90 (s, 1H), 7.75 (d, 1H), 7.60 (t, 1H), 7.40 (d, 1H), 7.22 (d, 1H), 6.85 (s, 1H), 2.90 (s, 3H), 2.36 (s, 3H), 1.78 (s, 6H). MS (ESI) m/z: 468.20 M + H)⁺ |
| 22 | 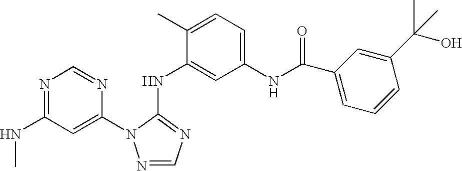 | ¹H NMR (400 MHz, DMSO) δ 8.70 (s, 1H), 8.55 (s, 1H), 8.05 (s, 1H), 7.95 (s, 1H), 7.90 (s, 1H), 7.80 (d, 1H), 7.78 (d, 1H), 7.45 (d, 1H), 7.40 (d, 1H), 7.20 (d, 1H), 6.85 (s, 1H), 2.90 (s, 3H), 2.36 (s, 3H), 1.50 (s, 6H). MS (ESI) m/z: 459.20 (M + H)⁺ |
| 23 | 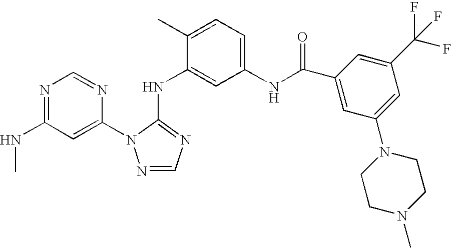 | MS (ESI) m/z: 567.2 (M + H)⁺ |
| 24 | 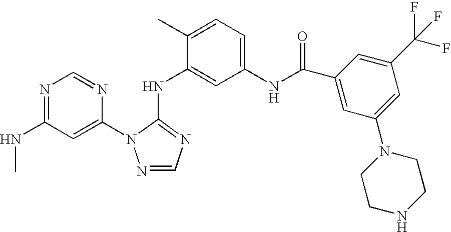 | MS (ESI) m/z: 553.2 (M + H)⁺ |
| 25 | 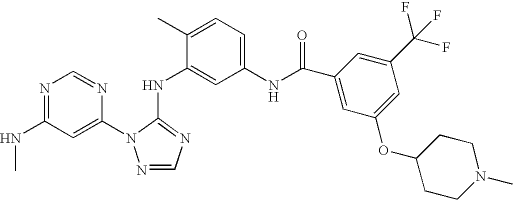 | MS (ESI) m/z: 582.2 (M + H)⁺ |
| 26 | 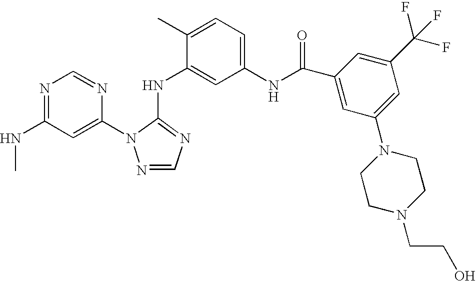 | MS (ESI) m/z: 597.2 (M + H)⁺ |
| 27 | 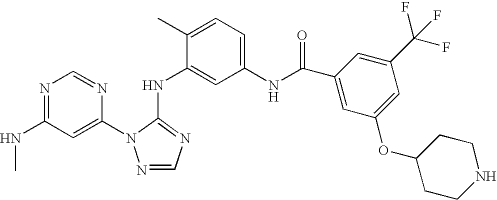 | MS (ESI) m/z: 568.2 (M + H)⁺ |
| 28 | 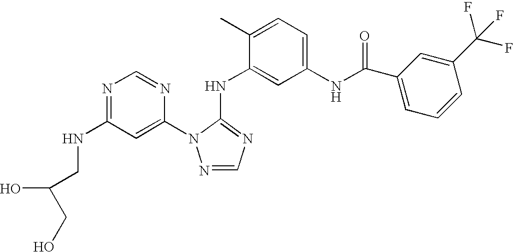 | MS (ESI) m/z: 530.3 (M + H)⁺ |
| 29 | 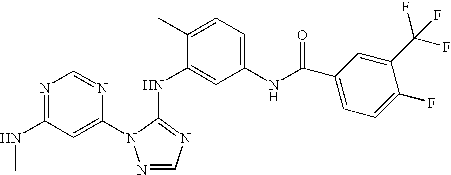 | MS (ESI) m/z: 487.0 (M + H)⁺ |
| 30 | 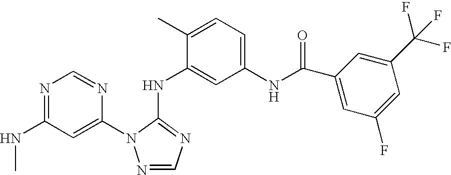 | MS (ESI) m/z: 487.1 (M + H)⁺ |
| 31 | 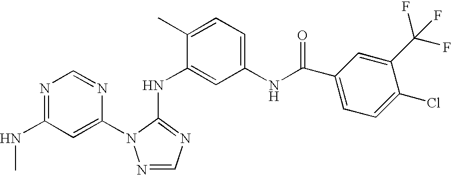 | MS (ESI) m/z: 503.1 (M + H)⁺ |
| 32 | 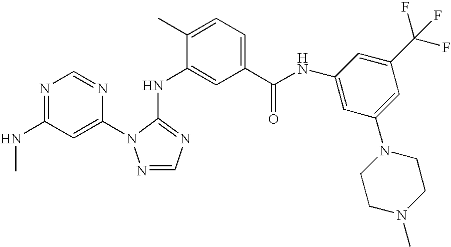 | MS (ESI) m/z: 567.2 (M + H)⁺ |
| 33 | 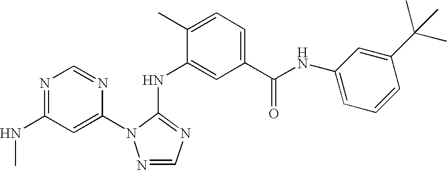 | MS (ESI) m/z: 457.20 (M + H)⁺ |
| 34 | 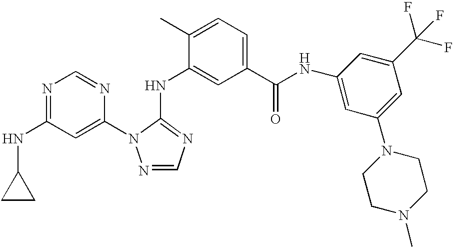 | MS (ESI) m/z: 593.2 (M + H)⁺ |
| 35 | 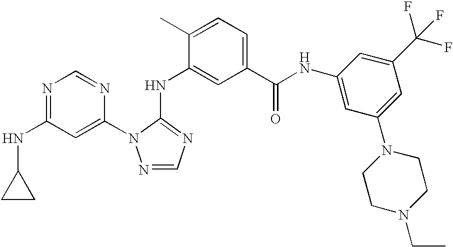 | MS (ESI) m/z: 607.2 (M + H)⁺ |
| 36 | 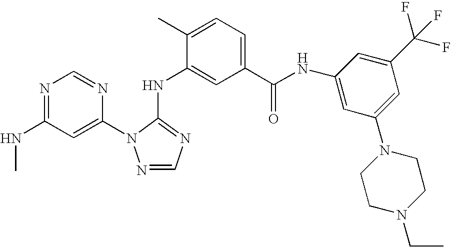 | MS (ESI) m/z: 581.2 (M + H)⁺ |
| 37 | 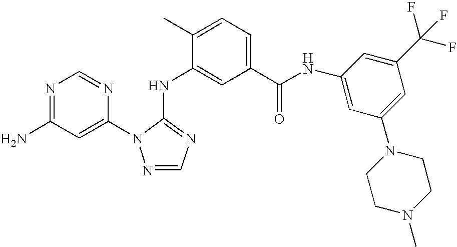 | MS (ESI) m/z: 553.2 (M + H)⁺ |
| 38 | 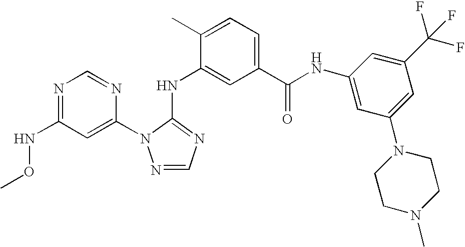 | MS (ESI) m/z: 583.10 (M + H)⁺ |
| 39 | 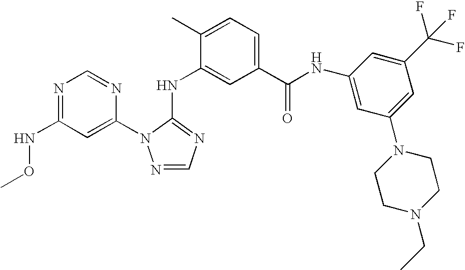 | MS (ESI) m/z: 597.20 (M + H)⁺ |
| 40 | 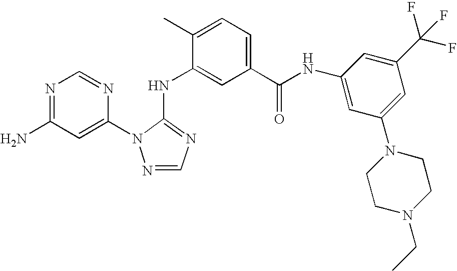 | MS (ESI) m/z: 567.2 (M + H)⁺ |
| 41 | 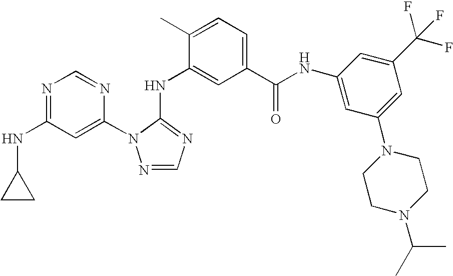 | MS (ESI) m/z: 621.3 (M + H)⁺ |
| 42 | 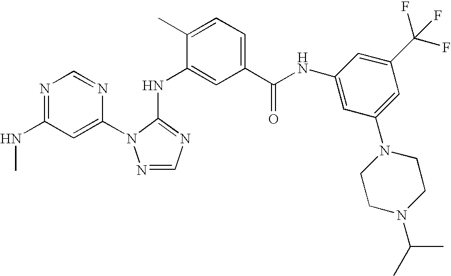 | MS (ESI) m/z: 595.20 (M + H)⁺ |
| 43 | 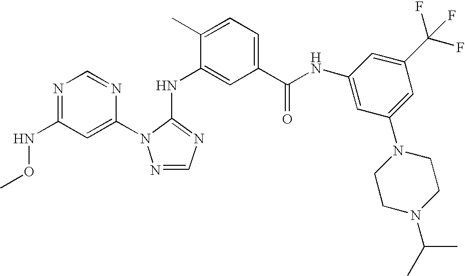 | MS (ESI) m/z: 611 (M + H)⁺ |
| 44 | 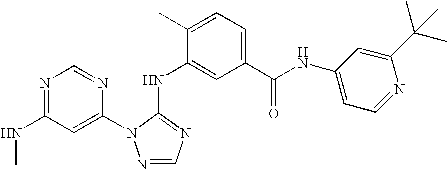 | MS (ESI) m/z: 491.1 (M + H)⁺ |
| 45 | 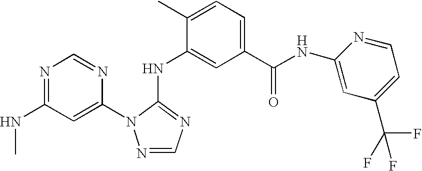 | MS (ESI) m/z: 470.1 (M + H)⁺ |
| 46 | 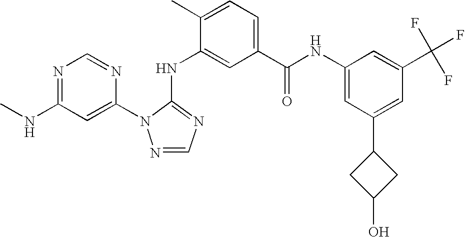 | MS (ESI) m/z: 539.20 (M + H)⁺ |
| 47 | 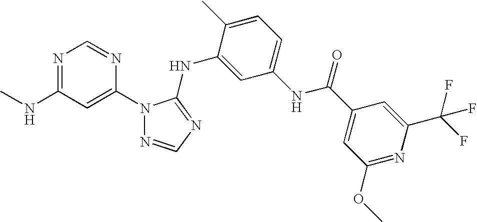 | MS (ESI) m/z: 500.2 (M + H)⁺ |
| 48 | 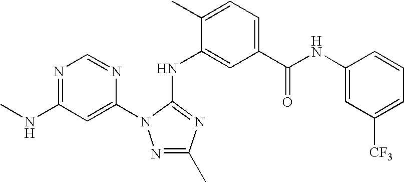 | MS (ESI) m/z: 483.1 (M + H)⁺ |
| 49 | 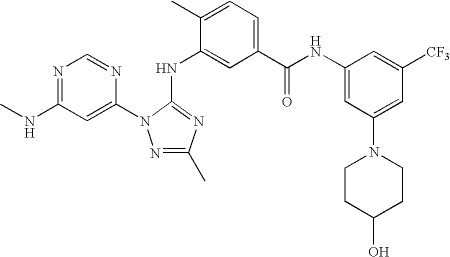 | MS (ESI) m/z: 582.2 (M + H)⁺ |
| 50 | 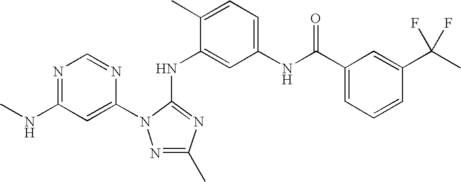 | MS (ESI) m/z: 479.2 (M + H)⁺ |
| 51 | 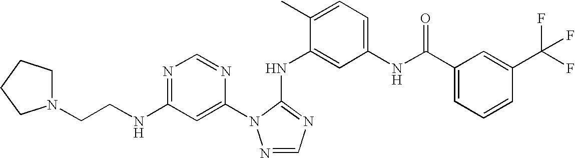 | MS (ESI) m/z: 552.2 (M + H)⁺ |
| 52 | 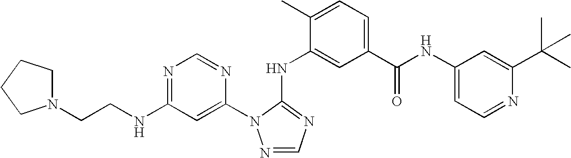 | MS (ESI) m/z: 541.3 (M + H)⁺ |
| 53 | 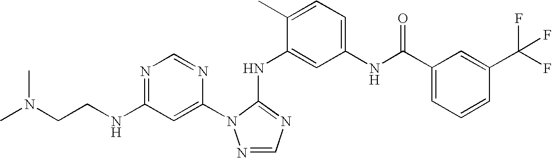 | MS (ESI) m/z: 526.2 (M + H)⁺ |
| 54 | 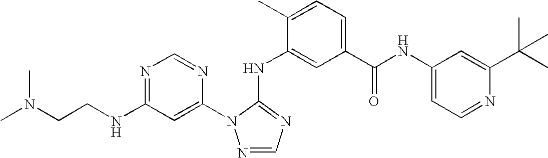 | MS (ESI) m/z: 515.2 (M + H)⁺ |
| 55 | 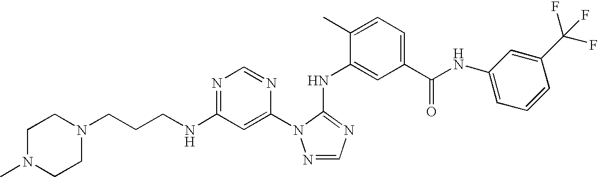 | MS (ESI) m/z: 595.2 (M + H)⁺ |
| 56 | 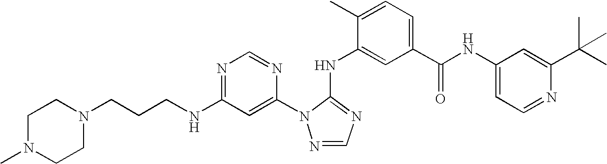 | MS (ESI) m/z: 584.3 (M + H)⁺ |
| 57 | 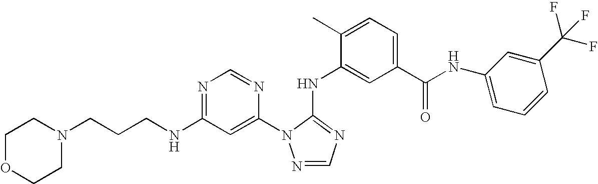 | MS (ESI) m/z: 582.2 (M + H)⁺ |
| 58 | 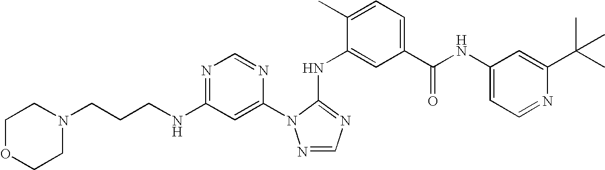 | MS (ESI) m/z: 571.3 (M + H)⁺ |
| 59 | 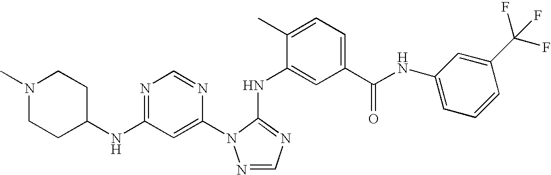 | MS (ESI) m/z: 552.2 (M + H)⁺ |
| 60 | 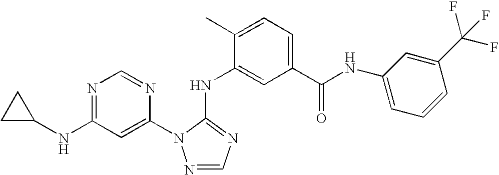 | MS (ESI) m/z: 495.2 (M + H)⁺ |
| 61 | 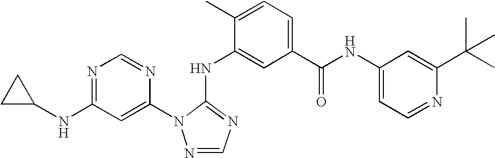 | MS (ESI) m/z: 484.2 (M + H)⁺ |
| 62 | 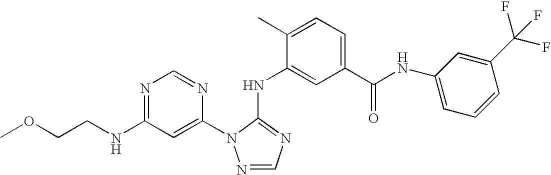 | MS (ESI) m/z: 513.2 (M + H)⁺ |
| 63 | 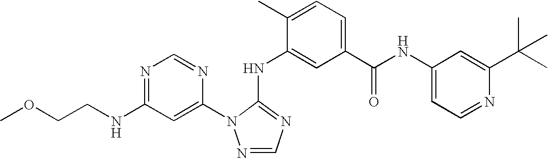 | MS (ESI) m/z: 502.2 (M + H)⁺ |
| 64 | 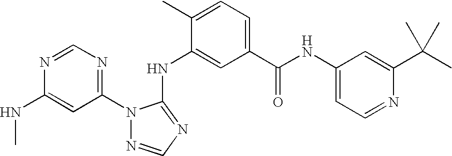 | MS (ESI) m/z: 458.2 (M + H)⁺ |
| 65 | 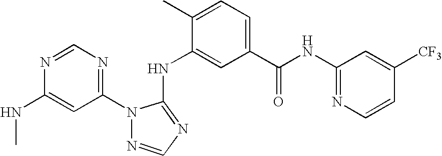 | MS (ESI) m/z: 470.1 (M + H)⁺ |
| 66 | 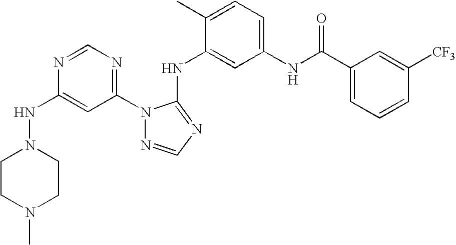 | MS (ESI) m/z: 553.2 (M + H)⁺ |
| 67 | 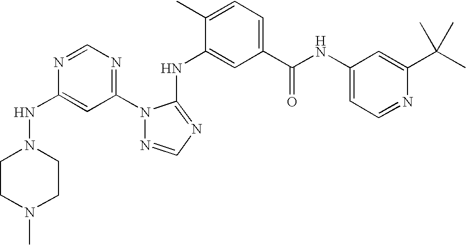 | MS (ESI) m/z: 542.3 (M + H)⁺ |
| 68 | 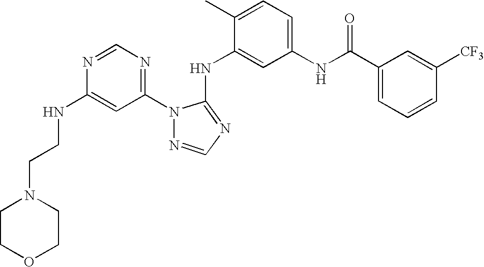 | MS (ESI) m/z: 568.2 (M + H)⁺ |
| 69 | 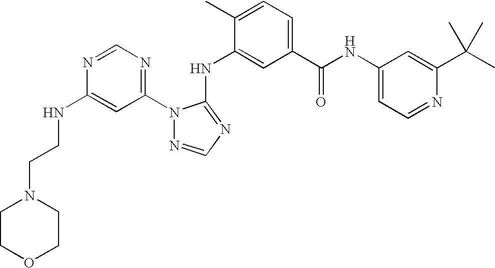 | MS (ESI) m/z: 557.3 (M + H)⁺ |
| 70 | 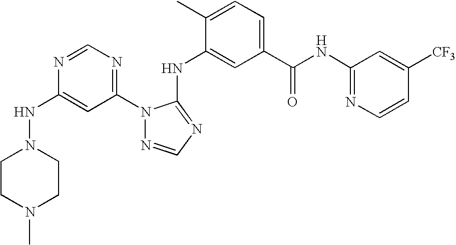 | MS (ESI) m/z: 554.2 (M + H)⁺ |
| 71 | 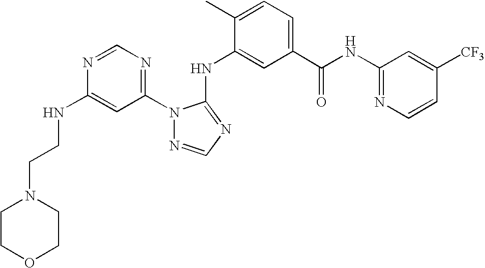 | MS (ESI) m/z: 569.2 (M + H)⁺ |
| 72 | 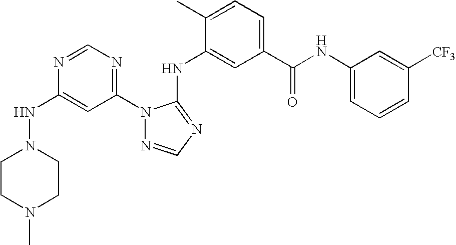 | MS (ESI) m/z: 553.2 (M + H)⁺ |
| 73 | 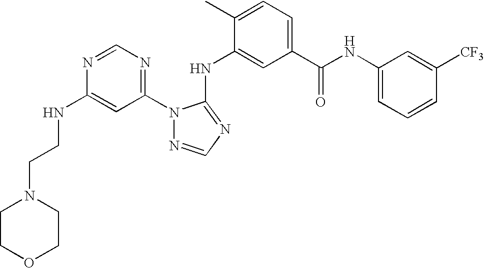 | MS (ESI) m/z: 568.2 (M + H)⁺ |
| 74 | 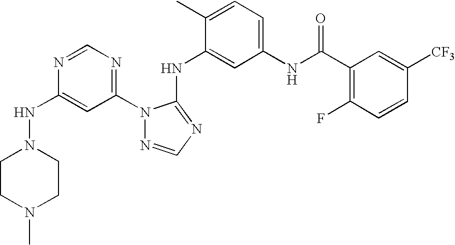 | MS (ESI) m/z: 571.2 (M + H)⁺ |
| 75 | 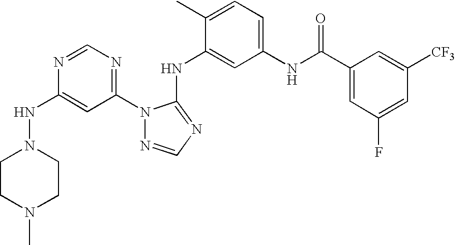 | MS (ESI) m/z: 571.2 (M + H)⁺ |
| 76 | 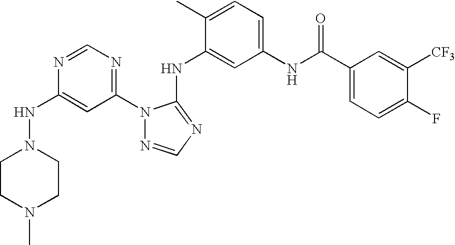 | MS (ESI) m/z: 571.2 (M + H)⁺ |
| 77 | 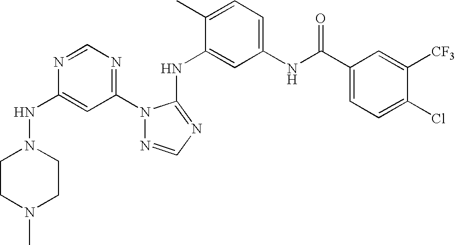 | MS (ESI) m/z: 587.2 (M + H)⁺ |
| 78 | 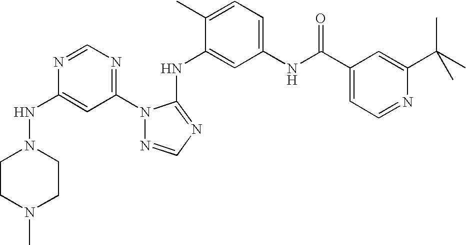 | MS (ESI) m/z: 542.3 (M + H)⁺ |
| 79 | 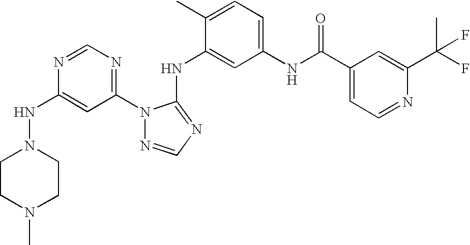 | MS (ESI) m/z: 550.2 (M + H)⁺ |
| 80 | 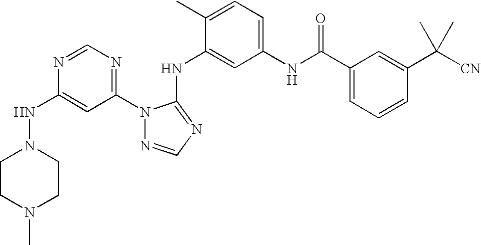 | MS (ESI) m/z: 552.3 (M + H)⁺ |
| 81 | 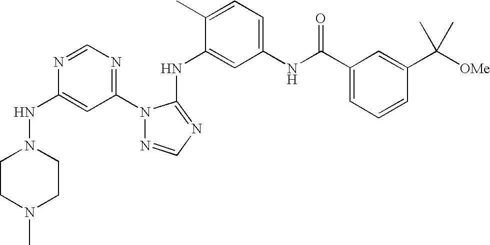 | MS (ESI) m/z: 557.3 (M + H)⁺ |
| 82 | 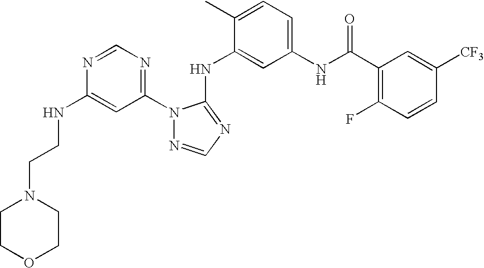 | MS (ESI) m/z: 586.2 (M + H)⁺ |
| 83 | 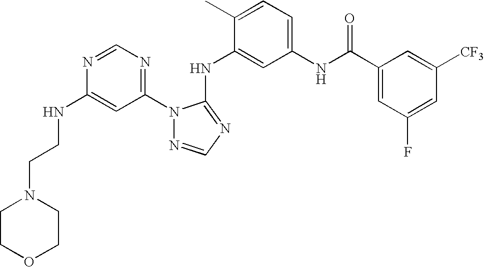 | MS (ESI) m/z: 586.2 (M + H)⁺ |
| 84 | 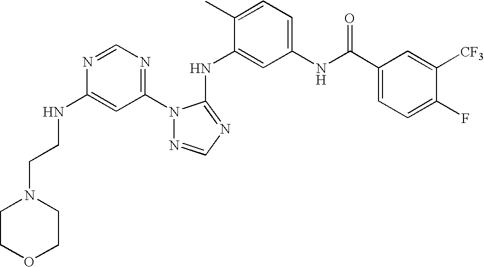 | MS (ESI) m/z: 586.2 (M + H)⁺ |
| 85 | 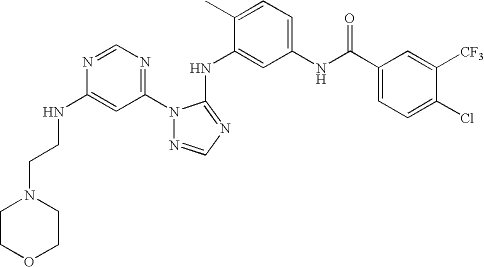 | MS (ESI) m/z: 602.2 (M + H)⁺ |
| 86 | 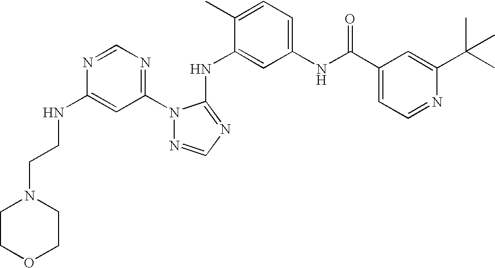 | MS (ESI) m/z: 557.3 (M + H)⁺ |
| 87 | 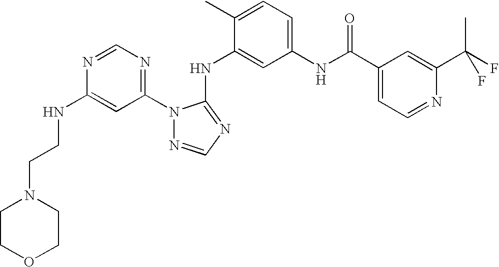 | MS (ESI) m/z: 565.2 (M + H)⁺ |
| 88 | 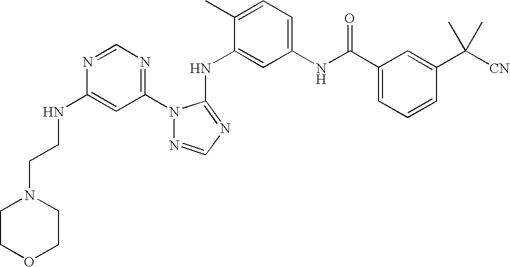 | MS (ESI) m/z: 567.3 (M + H)⁺ |
| 89 | 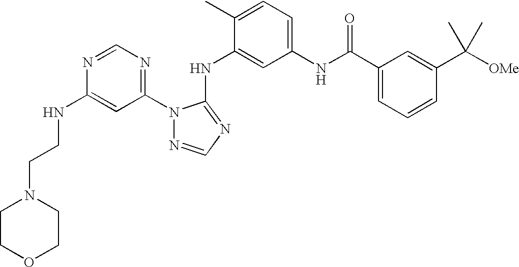 | MS (ESI) m/z: 572.3 (M + H)⁺ |
| 90 | 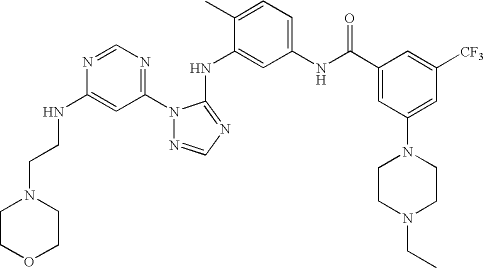 | MS (ESI) m/z: 680.4 (M + H)⁺ |
| 91 | 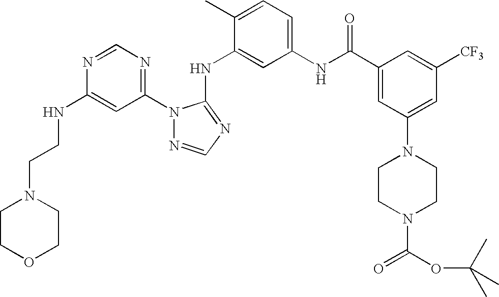 | MS (ESI) m/z: 752.4 (M + H)⁺ |
| 92 | 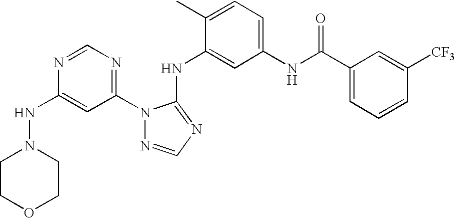 | MS (ESI) m/z: 540.2 (M + H)⁺ |
| 93 | 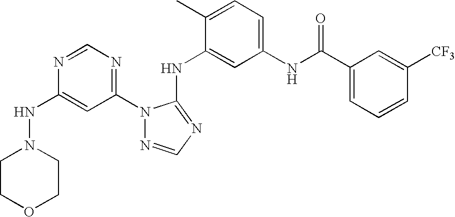 | MS (ESI) m/z: 540.2 (M + H)⁺ |
| 94 | 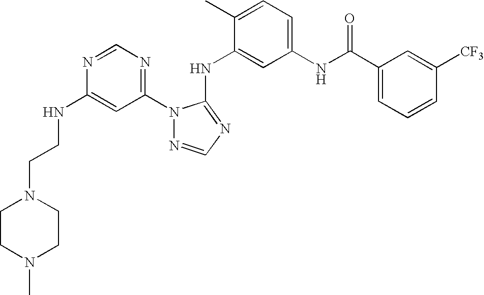 | MS (ESI) m/z: 581.3 (M + H)⁺ |
| 95 | 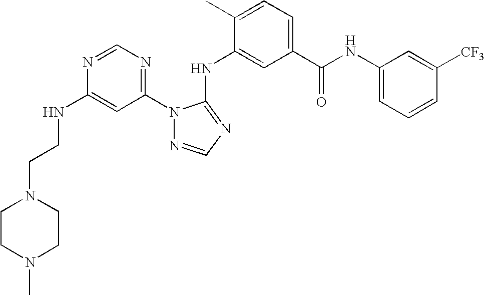 | MS (ESI) m/z: 581.3 (M + H)⁺ |
| 96 | 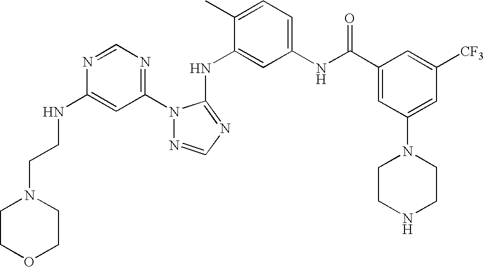 | MS (ESI) m/z: 652.3 (M + H)⁺ |
| 97 | 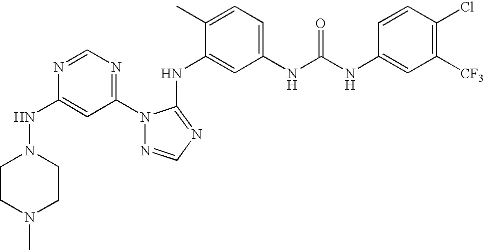 | MS (ESI) m/z: 602.2 (M + H)⁺ |
| 98 | 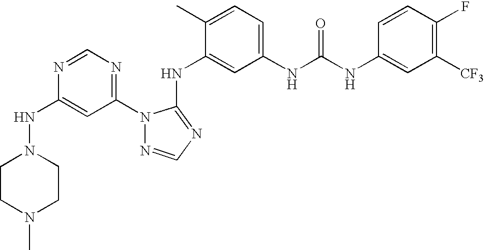 | MS (ESI) m/z: 586.2 (M + H)⁺ |
| 99 | 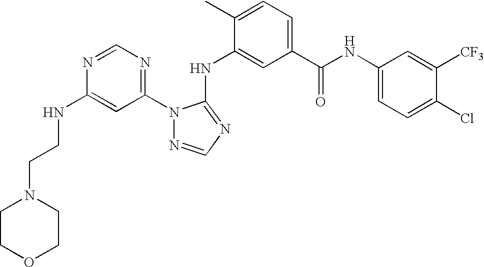 | MS (ESI) m/z: 602.2 (M + H)⁺ |
| 100 | 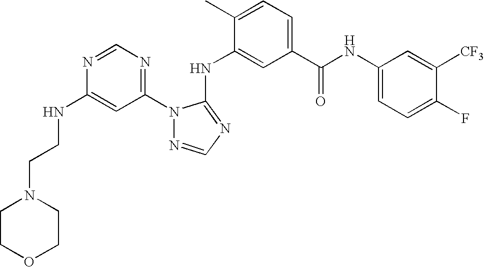 | MS (ESI) m/z: 586.2 (M + H)⁺ |
| 101 | 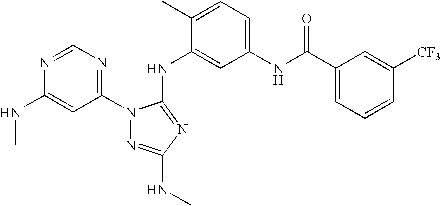 | MS (ESI) m/z: 498.2 (M + H)⁺ |
| 102 | 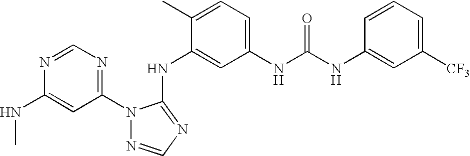 | MS (ESI) m/z: 484.1 (M + H)⁺ |
| 103 | 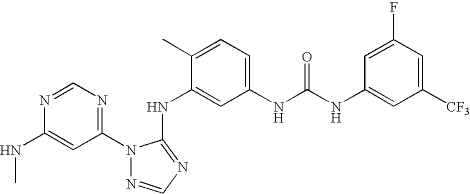 | MS (ESI) m/z: 502.1 (M + H)⁺ |
| 104 | 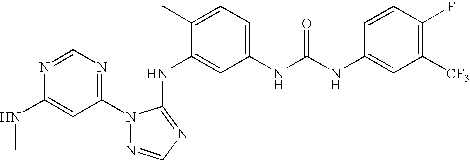 | MS (ESI) m/z: 502.1 (M + H)⁺ |
| 105 | 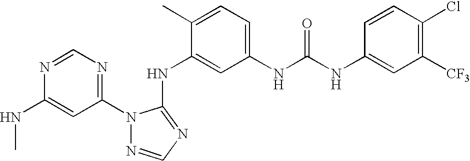 | MS (ESI) m/z: 518.1 (M + H)⁺ |
| 106 | 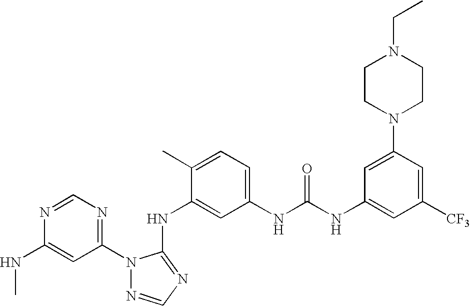 | MS (ESI) m/z: 596.3 (M + H)⁺ |
| 107 | 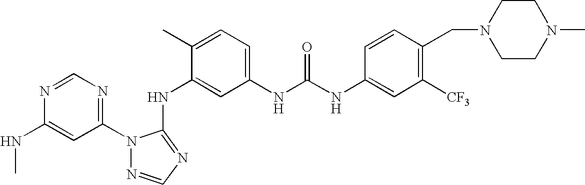 | MS (ESI) m/z: 596.3 (M + H)⁺ |
| 108 | 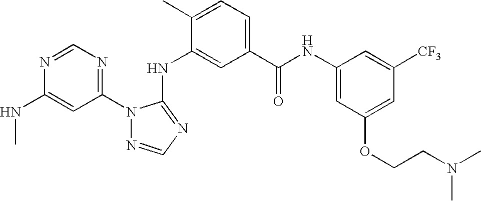 | MS (ESI) m/z: 556.2 (M + H)⁺ |
| 109 | 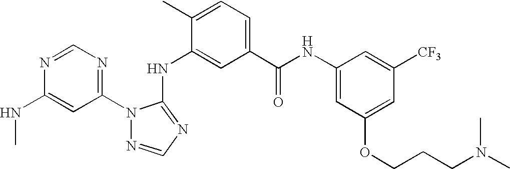 | MS (ESI) m/z: 570.3 (M + H)⁺ |
| 110 | 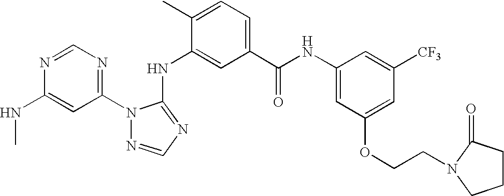 | MS (ESI) m/z: 596.2 (M + H)⁺ |
| 111 | 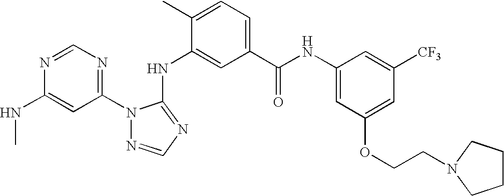 | MS (ESI) m/z: 582.3 (M + H)⁺ |
| 112 | 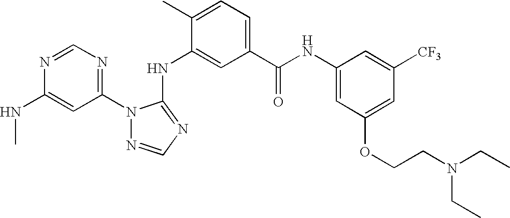 | MS (ESI) m/z: 584.3 (M + H)⁺ |
| 113 | 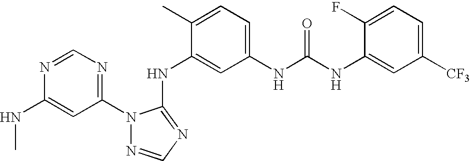 | MS (ESI) m/z: 502.2 (M + H)⁺ |
| 114 | 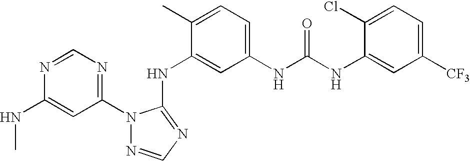 | MS (ESI) m/z: 518.1 (M + H)⁺ |
| 115 | 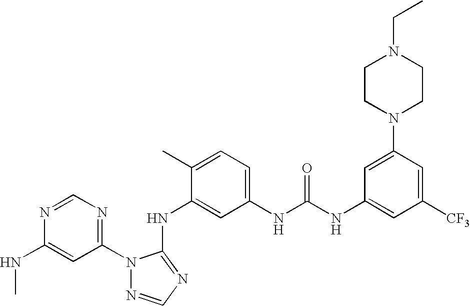 | MS (ESI) m/z: 596.3 (M + H)⁺ |
| 116 | 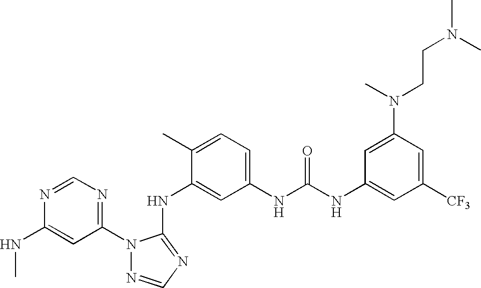 | MS (ESI) m/z: 584.3 (M + H)⁺ |
| 117 | 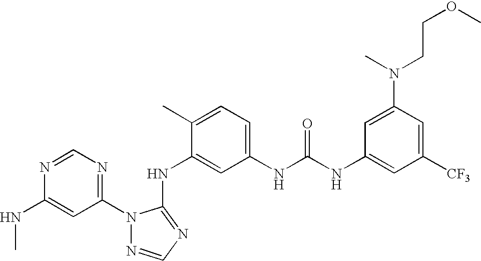 | MS (ESI) m/z: 571.2 (M + H)⁺ |
| 118 | 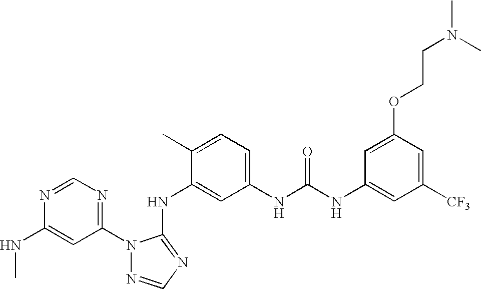 | MS (ESI) m/z: 571.2 (M + H)⁺ |
| 119 | 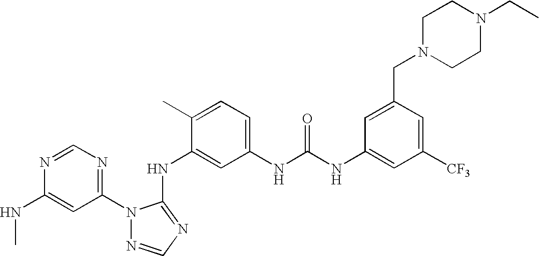 | MS (ESI) m/z: 610.3 (M + H)⁺ |
| 120 | 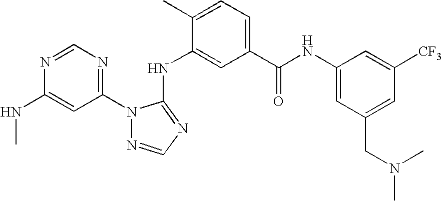 | MS (ESI) m/z: 526.2 (M + H)⁺ |
| 121 | 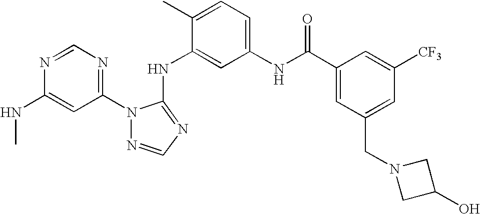 | MS (ESI) m/z: 553.5 (M + H)⁺ |

Assays

Compounds of the present invention may be assayed to measure their capacity to inhibit a kinase panel, including but not limited to Alk, Abl, Aurora-A, B-Raf, C-Raf, Bcr-Abl, BRK, Blk, Bmx, BTK, C-Kit, C-Src, EphB1, EphB2, EphB4, FLT1, Fms, Flt3, Fyn, FRK3, JAK2, KDR, Lck, Lyn, PDGFRα, PDGFRβ, PKCα, p38 (p38 MAP kinase, SAPK2α), Src, SIK, Syk, Tie2 and TrkB kinases.

B-Raf (Enzymatic Assay)

Compounds of the invention may be tested for their ability to inhibit the activity of B-Raf. The assay is carried out in 384-well MaxiSorp plates (NUNC) with black walls and clear bottom. The substrate, IκBα is diluted in DPBS (1:750) and 15 μl is added to each well. The plates are incubated at 4° C. overnight and washed 3 times with TBST (25 mM Tris, pH 8.0, 150 mM NaCl and 0.05% Tween-20) using the EMBLA plate washer. Plates are blocked by Superblock (15 μl/well) for 3 hours at room temperature, washed 3 times with TBST and pat-dried. Assay buffer containing 20 μM ATP (10 μl) is added to each well followed by 100 nl or 500 nl of compound. B-Raf is diluted in the assay buffer (1 μl into 25 μl) and 10 μl of diluted b-Raf is added to each well (0.4 μg/well). The plates are incubated at room temperature for 2.5 hours. The kinase reaction is stopped by washing the plates 6 times with TBST. Phosph-IκBα (Ser32/36) antibody is diluted in Superblock (1:10,000) and 15 μl is added to each well. The plates are incubated at 4° C. overnight and washed 6 times with TBST. AP-conjugated goat-anti-mouse IgG is diluted in Superblock (1:1,500) and 15 μl is added to each well. Plates are incubated at room temperature for 1 hour and washed 6 times with TBST. 15 μl of fluorescent Attophos AP substrate (Promega) is added to each well and plates are incubated at room temperature for 15 minutes. Plates are read on Acquest or Analyst GT using a Fluorescence Intensity Program (Excitation 455 nm, Emission 580 nm).

B-Raf (Cellular Assay)

Compounds of the invention are tested in A375 cells for their ability to inhibit phosphorylation of MEK. A375 cell line (ATCC) is derived from a human melanoma patient and has a V599E mutation on the B-Raf gene. The levels of phosphorylated MEK are elevated due to the mutation of B-Raf. Sub-confluent to confluent A375 cells are incubated with compounds for 2 hours at 37° C. in serum free medium. Cells are then washed once with cold PBS and lysed with the lysis buffer containing 1% Triton X100. After centrifugation, the supernatants are subjected to SDS-PAGE, and then transferred to nitrocellulose membranes. The membranes are then subjected to western blotting with anti-phospho-MEK antibody (ser217/221) (Cell Signaling). The amount of phosphorylated MEK is monitored by the density of phospho-MEK bands on the nitrocellulose membranes.

Inhibition of Cellular Bcr-Abl Dependent Proliferation (High Throughput Method)

The murine cell line 32D hemopoietic progenitor cell line may be transformed with Bcr-Abl cDNA (32D-p210). These cells are maintained in RPMI/10% fetal calf serum (RPMI/FCS) supplemented with penicillin 50 μg/mL, streptomycin 50 μg/mL and L-glutamine 200 mM. Untransformed 32D cells are similarly maintained with the addition of 15% of WEHI conditioned medium as a source of IL3.

50 μl of a 32D or 32D-p210 cells suspension are plated in Greiner 384 well microplates (black) at a density of 5000 cells per well. 50 nl of test compound (1 mM in DMSO stock solution) is added to each well (STI571 is included as a positive control). The cells are incubated for 72 hours at 37° C., 5% CO₂. 10 μl of a 60% Alamar Blue solution (Tek diagnostics) is added to each well and the cells are incubated for an additional 24 hours. The fluorescence intensity (Excitation at 530 nm, Emission at 580 nm) is quantified using the Acquest™ system (Molecular Devices).

Inhibition of Cellular Bcr-Abl Dependent Proliferation

32D-p210 cells are plated into 96 well TC plates at a density of 15,000 cells per well. 50 μL of two fold serial dilutions of the test compound (Cₘₐₓ is 40 μM) are added to each well (STI571 is included as a positive control). After incubating the cells for 48 hours at 37° C., 5% CO₂, 15 μL of MTT (Promega) is added to each well and the cells are incubated for an additional 5 hours. The optical density at 570 nm is quantified spectrophotometrically and IC₅₀ values, the concentration of compound required for 50% inhibition, determined from a dose response curve.

Effect on Cell Cycle Distribution

32D and 32D-p210 cells are plated into 96 well TC plates at 2.5×10⁶ cells per well in 5 ml of medium and test compound at 1 or 10 μM is added (STI571 is included as a control). The cells are then incubated for 24 or 48 hours at 37° C., 5% CO₂. 2 ml of cell suspension is washed with PBS, fixed in 70% EtOH for 1 hour and treated with PBS/EDTA/RNase A for 30 minutes. Propidium iodide (Cf=10 μg/ml) is added and the fluorescence intensity is quantified by flow cytometry on the FACScalibur™ system (BD Biosciences). In some embodiments, test compounds of the present invention may demonstrate an apoptotic effect on the 32D-p210 cells but not induce apoptosis in the 32D parental cells.

Effect on Cellular Bcr-Abl Autophosphorylation

Bcr-Abl autophosphorylation is quantified with capture Elisa using a c-Abl specific capture antibody and an antiphosphotyrosine antibody. 32D-p210 cells are plated in 96 well TC plates at 2×10⁵ cells per well in 50 μL of medium. 50 μL of two fold serial dilutions of test compounds (Cₘₐₓ is 10 μM) are added to each well (STI571 is included as a positive control). The cells are incubated for 90 minutes at 37° C., 5% CO₂. The cells are then treated for 1 hour on ice with 150 μL of lysis buffer (50 mM Tris-HCl, pH 7.4, 150 mM NaCl, 5 mM EDTA, 1 mM EGTA and 1% NP-40) containing protease and phosphatase inhibitors. 50 μL of cell lysate is added to 96 well optiplates previously coated with anti-Abl specific antibody and blocked. The plates are incubated for 4 hours at 4° C. After washing with TBS-Tween 20 buffer, 50 μL of alkaline-phosphatase conjugated anti-phosphotyrosine antibody is added and the plate is further incubated overnight at 4° C. After washing with TBS-Tween 20 buffer, 90 μL of a luminescent substrate are added and the luminescence is quantified using the Acquest™ system (Molecular Devices). In some embodiments, test compounds of the invention may inhibit the proliferation of the Bcr-Abl expressing cells, inhibiting the cellular Bcr-Abl autophosphorylation in a dose-dependent manner.

Effect on Proliferation of Cells Expressing Mutant Forms of Bcr-Abl

Compounds of the invention may be tested for their antiproliferative effect on Ba/F3 cells expressing either wild type or the mutant forms of Bcr-Abl (G250E, E255V, T315I, F317L, M351T) that confers resistance or diminished sensitivity to STI571. The antiproliferative effect of these compounds on the mutant-Bcr-Abl expressing cells and on the non transformed cells may be tested at 10, 3.3, 1.1 and 0.37 μM as described above (in media lacking IL3). The IC₅₀ values of the compounds lacking toxicity on the untransformed cells are determined from the dose response curves obtained as described above.

FGFR-3 (Enzymatic Assay)

Kinase activity assay with purified FGFR-3 (Upstate) is carried out in a final volume of 10 μL containing 0.25 μg/mL of enzyme in kinase buffer (30 mM Tris-HCl pH7.5, 15 mM MgCl₂, 4.5 mM MnCl₂, 15 μM Na₃VO₄ and 50 μg/mL BSA), and substrates (5 μg/mL biotin-poly-EY(Glu, Tyr) (CIS-US, Inc.) and 3 μM ATP). Two solutions are made: the first solution of 5 μl contains the FGFR-3 enzyme in kinase buffer was first dispensed into 384-well format ProxiPlate® (Perkin-Elmer) followed by adding 50 nL of compounds dissolved in DMSO, then 5 of second solution contains the substrate (poly-EY) and ATP in kinase buffer was added to each well. The reactions are incubated at room temperature for one hour, stopped by adding 10 μL of HTRF detection mixture, which contains 30 mM Tris-HCl pH 7.5, 0.5 M KF, 50 mM ETDA, 0.2 mg/mL BSA, 15 μg/mL streptavidin-XL665 (CIS-US, Inc.) and 150 ng/mL cryptate conjugated anti-phosphotyrosine antibody (CIS-US, Inc.). After one hour of room temperature incubation to allow for streptavidin-biotin interaction, time resolved florescent signals are read on Analyst GT (Molecular Devices Corp.). IC₅₀ values are calculated by linear regression analysis of the percentage inhibition of each compound at 12 concentrations (1:3 dilution from 50 μM to 0.28 nM). In this assay, compounds of the invention have an IC₅₀ in the range of 10 nM to 2 μM.

FGFR-3 (Cellular Assay)

Compounds of the invention are tested for their ability to inhibit transformed Ba/F3-TEL-FGFR-3 cells proliferation, which is dependent on FGFR-3 cellular kinase activity. Ba/F3-TEL-FGFR-3 are cultured up to 800,000 cells/mL in suspension, with RPMI 1640 supplemented with 10% fetal bovine serum as the culture medium. Cells are dispensed into 384-well format plate at 5000 cell/well in 50 μL culture medium. Compounds of the invention are dissolved and diluted in dimethylsulfoxide (DMSO). Twelve points 1:3 serial dilutions are made into DMSO to create concentrations gradient ranging typically from 10 mM to 0.05 μM. Cells are added with 50 nL of diluted compounds and incubated for 48 hours in cell culture incubator. AlamarBlue® (TREK Diagnostic Systems), which can be used to monitor the reducing environment created by proliferating cells, are added to cells at a final concentration of 10%. After additional four hours of incubation in a 37° C. cell culture incubator, fluorescence signals from reduced AlamarBlue® (Excitation at 530 nm, Emission at 580 nm) are quantified on Analyst GT (Molecular Devices Corp.). IC₅₀ values are calculated by linear regression analysis of the percentage inhibition of each compound at 12 concentrations.

FLT3 and PDGFRβ

The effects of compounds of the invention on the cellular activity of FLT3 and PDGFRβ may be conducted following identical methods as described above for FGFR3 cellular activity, using Ba/F3-FLT3-ITD and Ba/F3-Tel-PDGFRβ.

Compounds of the invention may be tested for their ability to inhibit transformed Ba/F3-FLT3-ITD or Ba/F3-Tel-PDGFRβ cells proliferation, which is dependent on FLT3 or PDGFRβ cellular kinase activity. Ba/F3-FLT3-ITD or Ba/F3-Tel-PDGFRβ are cultured up to 800,000 cells/mL in suspension, with RPMI 1640 supplemented with 10% fetal bovine serum as the culture medium. Cells are dispensed into 384-well format plate at 5000 cell/well in 50 μL culture medium. Compounds of the invention are dissolved and diluted in dimethylsulfoxide (DMSO). Twelve points 1:3 serial dilutions are made into DMSO to create concentrations gradient ranging typically from 10 mM to 0.05 μM. Cells are added with 50 nL of diluted compounds and incubated for 48 hours in cell culture incubator. AlamarBlue® (TREK Diagnostic Systems), which can be used to monitor the reducing environment created by proliferating cells, are added to cells at final concentration of 10%. After additional four hours of incubation in a 37° C. cell culture incubator, fluorescence signals from reduced AlamarBlue® (Excitation at 530 nm, Emission at 580 nm) are quantified on Analyst GT (Molecular Devices Corp.). IC₅₀ values are calculated by linear regression analysis of the percentage inhibition of each compound at 12 concentrations.

C-Kit

Compounds of the invention may be tested for inhibition of SCF dependent proliferation using Mo7e cells which endogenously express c-Kit in a 96-well format. Briefly, two-fold serially diluted test compounds (Cₘₐₓ=10 μM) are evaluated for their antiproliferative activity on Mo7e cells stimulated with human recombinant SCF. After 48 hour incubation at 37° C., cell viability is measured by using a MTT colorimetric assay from Promega.

Upstate KinaseProfiler™ Radio-Enzymatic Filter Binding Assay

Compounds of the invention may be assessed for their ability to inhibit individual member of a panel of kinases (a partial, non-limiting list of kinases includes: Alk, Abl, Aurora-A, B-Raf, Bcr-Abl, BRK, Blk, Bmx, C-Kit, C-Raf, C-Src, CSK, EphB, FLT1, Fms, Fyn, JAK2, KDR, Lck, Lyn, PDGFRα, PDGFRβ, PKCα, p38 (p38 MAP kinase, SAPK2α), SIK, Src, Syk, Tie2 and TrkB kinases). The compounds are tested in duplicates at a final concentration of 10 μM following this generic protocol, using varying kinase buffer composition and substrates for the different kinases included in the “Upstate KinaseProfiler™ panel. Kinase buffer (2.5 μL, 10×—containing MnCl₂ when required), active kinase (0.001-0.01 Units; 2.5 μL), specific or Poly(Glu4-Tyr) peptide (5-500 μM or 0.01 mg/ml) in kinase buffer and kinase buffer (50 μM; 5 μL) are mixed in an eppendorf on ice. A Mg/ATP mix (10 μL; 67.5 (or 33.75) mM MgCl₂, 450 (or 225) μM ATP and 1 μCi/μl [γ-³²P]-ATP (3000 Ci/mmol)) is added and the reaction is incubated at about 30° C. for about 10 minutes. The reaction mixture is spotted (20 μL) onto a 2 cm×2 cm P81 (phosphocellulose, for positively charged peptide substrates) or Whatman No. 1 (for Poly(Glu4-Tyr) peptide substrate) paper square. The assay squares are washed 4 times, for 5 minutes each, with 0.75% phosphoric acid and washed once with acetone for 5 minutes. The assay squares are transferred to a scintillation vial, 5 ml scintillation cocktail are added and ³²P incorporation (cpm) to the peptide substrate is quantified with a Beckman scintillation counter. Percentage inhibition is calculated for each reaction.

Compounds of Formula (1), (2) or (3) in free form or in pharmaceutically acceptable salt form, may exhibit valuable pharmacological properties, for example, as indicated by the in vitro tests described in this application. The IC₅₀ value in those experiments is given as that concentration of the test compound in question that results in a cell count that is 50% lower than that obtained using the control without inhibitor. In general, compounds of the invention have IC₅₀ values from 1 nM to 10 μM against one or more of the following kinases: Alk, Abl, Aurora-A, B-Raf, C-Raf, Bcr-Abl, BRK, Blk, Bmx, BTK, C-Kit, C-Src, EphB1, EphB2, EphB4, FLT1, Fms, Flt3, Fyn, FRK3, JAK2, KDR, Lck, Lyn, PDGFRα, PDGFRβ, PKCα, p38 (p38 MAP kinase, SAPK2α), Src, SIK, Syk, Tie2 and TrkB kinases.

In some examples, compounds of the invention have IC₅₀ values from 0.01 μM to 5 μM. In other examples, compounds of the invention have IC₅₀ values from 0.01 μM to 1 μM, or more particularly from 1 nM to 1 μM. In yet other examples, compounds of the invention have IC₅₀ values of <0-100 nM, 100-250 nM, 250-500 nM, or >500 nM. Compounds of the invention may also have IC₅₀ values of less than 1 nM or more than 10 μM.

Compounds of Formula (1), (2) or (3) may exhibit a percentage inhibition of greater than 50%, or in other embodiments, may exhibit a percentage inhibition greater than about 70%, against one or more of the following kinases at 10 μM: Alk, Abl, Aurora-A, B-Raf, C-Raf, Bcr-Abl, BRK, Blk, Bmx, BTK, C-Kit, C-Src, EphB1, EphB2, EphB4, FLT1, Fms, Flt3, Fyn, FRK3, JAK2, KDR, Lck, Lyn, PDGFRα, PDGFRβ, PKCα, p38 (p38 MAP kinase, SAPK2α), Src, SIK, Syk, Tie2 and TrkB kinases.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

1. A compound of Formula (1): 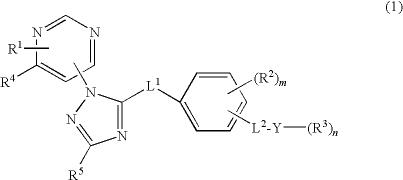
or pharmaceutically acceptable salts thereof, wherein:
L¹ is NR, NRCO or NRSO₁₋₂;
L² are independently NRCO, NRCONR, CONR, NRSO₁₋₂ or SO₁₋₂NR;
Y is a C₃₋₇ cycloalkyl, C₃₋₇ heterocycloalkyl, or a monocyclic or fused C₆₋₁₀ aryl or 5-10 membered heteroaryl having 1-3 heteroatoms selected from N, O and S;
R¹ and R⁵ are independently H, an optionally halogenated C₁₋₆ alkyl, NR₂ or halo;
R² is an optionally halogenated C₁₋₆ alkyl or halo;
R³ is halo, substituted or unsubstituted C₁₋₆ alkyl, C₂₋₆ alkenyl, or C₂₋₆ alkynyl; optionally halogenated C₁₋₆ alkoxy, XR⁸, XO(CR₂)ₚR⁹, O(CR₂)ₚNR⁶R⁷, XNR⁶R⁷ or XNR(CR₂)ₚNR⁶R⁷;
R⁴ is NR⁶R⁷, NR(CR₂)ₚNR⁶R⁷, NRCONR⁶R⁷ or NRCO₂R⁶;
R⁶ and R⁷ are independently H, an optionally halogenated C₁₋₆ alkyl, C₂₋₆ alkenyl or C₂₋₆ alkynyl; C₁₋₆ alkanol, XR⁸ or XO(CR₂)ₚR⁹; or R⁶ and R⁷ together with N in NR⁶R⁷ may form an optionally substituted ring;
R⁸ and R⁹ are independently an optionally substituted C₃₋₇ cycloalkyl, C₆₋₁₀ aryl or 5-7 membered heterocyclic or heteroaryl having 1-3 heteroatoms selected from N, O and S; or R⁹ is H;
each R is H or C₁₋₆ alkyl;
each X is a bond or a C₁₋₄ alkylene;
m is 0-2; and
n and p are independently 0-4.

2. The compound of claim 1, wherein L¹ is NH.

3. The compound of claim 1, wherein L² is NHCO, CONH, or NHCONH.

4. The compound of claim 1, wherein Y is a monocyclic or fused C₆₋₁₀ aryl or a 5-10 membered heteroaryl having 1-3 heteroatoms selected from N, O and S.

5. The compound of claim 4, wherein Y is phenyl, pyridyl, thienyl, pyrazolyl, isoxazolyl, furanyl or pyrrolyl.

6. The compound of claim 1, wherein R¹ is H.

7. The compound of claim 1, wherein R² is CH₃.

8. The compound of claim 1, wherein R³ is halo, C₁₋₆ alkyl, optionally substituted with halo, hydroxyl, alkoxy, or cyano; optionally halogenated C₁₋₆ alkoxy, XR⁸, XO(CR₂)ₚR⁹, O(CR₂)ₚNR⁶R⁷, XNR⁶R⁷ or XNR(CR₂)ₚNR⁶R⁷.

9. The compound of claim 1, wherein R⁶ and R⁷ together with N form an optionally substituted piperidinyl, morpholinyl, piperazinyl, pyrrolidinyl, pyrrolidonyl or imidazolyl.

10. The compound of claim 1, wherein R⁸ and R⁹ are independently an optionally substituted C₃₋₇ cycloalkyl, piperidinyl, morpholinyl, piperazinyl, pyrrolidinyl, pyrrolidonyl, imidazolyl, pyridyl, phenyl, furanyl, naphthalenyl, pyrimidinyl, triazolyl, isothiazolyl, isoxazolyl, pyrazolyl or pyrazinyl.

11. The compound of claim 1, wherein said compound is of Formula (2) or (3):

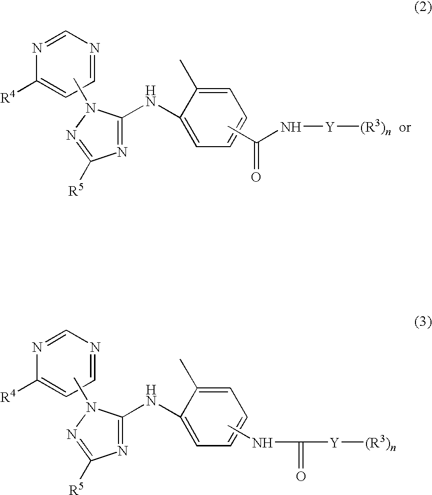

12. The compound of claim 1, wherein each optionally substituted ring is optionally substituted with halo, optionally halogenated C₁₋₆ alkyl, C₂₋₆ alkenyl, or C₂₋₆ alkynyl; nitro, cyano, XCO₂R¹⁰, XOR¹⁰, XR⁸, XNRCOR¹⁰, XNR₂, XNSO₁₋₂R, XNRSR, XNRCONR₂, XNR(CR₂)ₚNR₂, XNR(CR₂)ₚOR, XNR(C═NR)NR₂, XCONR₂, XCONR(CR₂)ₚNR₂, XSO₂R⁸, XSO₁₋₂NR₂ or XSNR₂; wherein R¹⁰ is H, optionally halogenated C₁₋₆ alkyl, C₃₋₇ cycloalkyl, C₆₋₁₀ aryl or a 5-7 membered heterocyclic or heteroaryl having 1-3 heteroatoms selected from N, O and S.

13. The compound of claim 1, wherein said compound is selected from the group consisting of:
N-{4-Methyl-3-[2-(6-methylamino-pyrimidin-4-yl)-2H-[1,2,4]triazol-3-ylamino]-phenyl}-3-trifluoromethyl-benzamide;
4-Methyl-3-[2-(6-methylamino-pyrimidin-4-yl)-2H-[1,2,4]triazol-3-ylamino]-N-(3-trifluoromethyl-phenyl)-benzamide;
4-Methyl-3-(3-methyl-1-(6-(methylamino)pyrimidin-4-yl)-1H-1,2,4-triazol-5-ylamino)-N-(3-(4-methylpiperazin-1-yl)-5-(trifluoromethyl)phenyl)benzamide;
1-{4-Methyl-3-[2-(6-methylamino-pyrimidin-4-yl)-2H-[1,2,4]triazol-3-ylamino]-phenyl}-3-(3-pyrrolidin-1-ylmethyl-5-trifluoromethyl-phenyl)-urea;
3-(1,1-difluoroethyl)-N-(4-methyl-3-(1-(6-(methylamino)pyrimidin-4-yl)-1H-1,2,4-triazol-5-ylamino)phenyl)benzamide;
2-tert-butyl-N-(4-methyl-3-(1-(6-(methylamino)pyrimidin-4-yl)-1H-1,2,4-triazol-5-ylamino)phenyl)isonicotinamide;
2-(2-hydroxypropan-2-yl)-N-(4-methyl-3-(1-(6-(methylamino)pyrimidin-4-yl)-1H-1,2,4-triazol-5-ylamino)phenyl)isonicotinamide;
2-(1,1-difluoroethyl)-N-(4-methyl-3-(1-(6-(methylamino)pyrimidin-4-yl)-1H-1,2,4-triazol-5-ylamino)phenyl)isonicotinamide;
3-(4-ethylpiperazin-1-yl)-N-(4-methyl-3-(1-(6-(methylamino)pyrimidin-4-yl)-1H-1,2,4-triazol-5-ylamino)phenyl)-5-(trifluoromethyl)benzamide;
3-isopropoxy-N-(4-methyl-3-(1-(6-(methylamino)pyrimidin-4-yl)-1H-1,2,4-triazol-5-ylamino)phenyl)benzamide;
N-(4-methyl-3-(1-(6-(methylamino)pyrimidin-4-yl)-1H-1,2,4-triazol-5-ylamino)phenyl)-3-(trifluoromethoxy)benzamide;
3-(2-cyanobutan-2-yl)-N-(4-methyl-3-(1-(6-(methylamino)pyrimidin-4-yl)-1H-1,2,4-triazol-5-ylamino)phenyl)benzamide;
3-(2-cyanopropan-2-yl)-N-(4-methyl-3-(1-(6-(methylamino)pyrimidin-4-yl)-1H-1,2,4-triazol-5-ylamino)phenyl)benzamide;
3-(2-hydroxypropan-2-yl)-N-(4-methyl-3-(1-(6-(methylamino)pyrimidin-4-yl)-1H-1,2,4-triazol-5-ylamino)phenyl)benzamide;
N-(4-methyl-3-(1-(6-(methylamino)pyrimidin-4-yl)-1H-1,2,4-triazol-5-ylamino)phenyl)-3-(4-methylpiperazin-1-yl)-5-(trifluoromethyl)benzamide;
N-(4-methyl-3-(1-(6-(methylamino)pyrimidin-4-yl)-1H-1,2,4-triazol-5-ylamino)phenyl)-3-(piperazin-1-yl)-5-(trifluoromethyl)benzamide;
N-(4-methyl-3-(1-(6-(methylamino)pyrimidin-4-yl)-1H-1,2,4-triazol-5-ylamino)phenyl)-3-(1-methylpiperidin-4-yloxy)-5-(trifluoromethyl)benzamide;
3-(4-(2-hydroxyethyl)piperazin-1-yl)-N-(4-methyl-3-(1-(6-(methylamino)pyrimidin-4-yl)-1H-1,2,4-triazol-5-ylamino)phenyl)-5-(trifluoromethyl)benzamide;
N-(4-methyl-3-(1-(6-(methylamino)pyrimidin-4-yl)-1H-1,2,4-triazol-5-ylamino)phenyl)-3-(piperidin-4-yloxy)-5-(trifluoromethyl)benzamide;
N-(3-(1-(6-(2,3-dihydroxypropylamino)pyrimidin-4-yl)-1H-1,2,4-triazol-5-ylamino)-4-methylphenyl)-3-(trifluoromethyl)benzamide;
4-fluoro-N-(4-methyl-3-(1-(6-(methylamino)pyrimidin-4-yl)-1H-1,2,4-triazol-5-ylamino)phenyl)-3-(trifluoromethyl)benzamide;
3-fluoro-N-(4-methyl-3-(1-(6-(methylamino)pyrimidin-4-yl)-1H-1,2,4-triazol-5-ylamino)phenyl)-5-(trifluoromethyl)benzamide;
4-chloro-N-(4-methyl-3-(1-(6-(methylamino)pyrimidin-4-yl)-1H-1,2,4-triazol-5-ylamino)phenyl)-3-(trifluoromethyl)benzamide;
4-methyl-3-(1-(6-(methylamino)pyrimidin-4-yl)-1H-1,2,4-triazol-5-ylamino)-N-(3-(4-methylpiperazin-1-yl)-5-(trifluoromethyl)phenyl)benzamide;
N-(3-tert-butylphenyl)-4-methyl-3-(1-(6-(methylamino)pyrimidin-4-yl)-1H-1,2,4-triazol-5-ylamino)benzamide;
3-(1-(6-(cyclopropylamino)pyrimidin-4-yl)-1H-1,2,4-triazol-5-ylamino)-4-methyl-N-(3-(4-methylpiperazin-1-yl)-5-(trifluoromethyl)phenyl)benzamide;
3-(1-(6-(cyclopropylamino)pyrimidin-4-yl)-1H-1,2,4-triazol-5-ylamino)-N-(3-(4-ethylpiperazin-1-yl)-5-(trifluoromethyl)phenyl)-4-methylbenzamide;
N-(3-(4-ethylpiperazin-1-yl)-5-(trifluoromethyl)phenyl)-4-methyl-3-(1-(6-(methylamino)pyrimidin-4-yl)-1H-1,2,4-triazol-5-ylamino)benzamide;
3-(1-(6-aminopyrimidin-4-yl)-1H-1,2,4-triazol-5-ylamino)-4-methyl-N-(3-(4-methylpiperazin-1-yl)-5-(trifluoromethyl)phenyl)benzamide;
3-(1-(6-(methoxyamino)pyrimidin-4-yl)-1H-1,2,4-triazol-5-ylamino)-4-methyl-N-(3-(4-methylpiperazin-1-yl)-5-(trifluoromethyl)phenyl)benzamide;
N-(3-(4-ethylpiperazin-1-yl)-5-(trifluoromethyl)phenyl)-3-(1-(6-(methoxyamino)pyrimidin-4-yl)-1H-1,2,4-triazol-5-ylamino)-4-methylbenzamide;
3-(1-(6-aminopyrimidin-4-yl)-1H-1,2,4-triazol-5-ylamino)-N-(3-(4-ethylpiperazin-1-yl)-5-(trifluoromethyl)phenyl)-4-methylbenzamide;
3-(1-(6-(cyclopropylamino)pyrimidin-4-yl)-1H-1,2,4-triazol-5-ylamino)-N-(3-(4-isopropylpiperazin-1-yl)-5-(trifluoromethyl)phenyl)-4-methylbenzamide;
N-(3-(4-isopropylpiperazin-1-yl)-5-(trifluoromethyl)phenyl)-4-methyl-3-(1-(6-(methylamino)pyrimidin-4-yl)-1H-1,2,4-triazol-5-ylamino)benzamide;
N-(3-(4-isopropylpiperazin-1-yl)-5-(trifluoromethyl)phenyl)-3-(1-(6-(methoxyamino)pyrimidin-4-yl)-1H-1,2,4-triazol-5-ylamino)-4-methylbenzamide;
N-(2-tert-butylpyridin-4-yl)-4-methyl-3-(1-(6-(methylamino)pyrimidin-4-yl)-1H-1,2,4-triazol-5-ylamino)benzamide;
4-methyl-3-(1-(6-(methylamino)pyrimidin-4-yl)-1H-1,2,4-triazol-5-ylamino)-N-(4-(trifluoromethyl)pyridin-2-yl)benzamide;
N-(3-(3-hydroxycyclobutyl)-5-(trifluoromethyl)phenyl)-4-methyl-3-(1-(6-(methylamino)pyrimidin-4-yl)-1H-1,2,4-triazol-5-ylamino)benzamide;
2-methoxy-N-(4-methyl-3-(1-(6-(methylamino)pyrimidin-4-yl)-1H-1,2,4-triazol-5-ylamino)phenyl)-6-(trifluoromethyl)isonicotinamide;
4-methyl-3-(3-methyl-1-(6-(methylamino)pyrimidin-4-yl)-1H-1,2,4-triazol-5-ylamino)-N-(3-(trifluoromethyl)phenyl)benzamide;
N-(3-(4-hydroxypiperidin-1-yl)-5-(trifluoromethyl)phenyl)-4-methyl-3-(3-methyl-1-(6-(methylamino)pyrimidin-4-yl)-1H-1,2,4-triazol-5-ylamino)benzamide;
3-(1,1-difluoroethyl)-N-(4-methyl-3-(3-methyl-1-(6-(methylamino)pyrimidin-4-yl)-1H-1,2,4-triazol-5-ylamino)phenyl)benzamide;
N-(4-methyl-3-(1-(6-(2-(pyrrolidin-1-yl)ethylamino)pyrimidin-4-yl)-1H-1,2,4-triazol-5-ylamino)phenyl)-3-(trifluoromethyl)benzamide;
N-(2-tert-butylpyridin-4-yl)-4-methyl-3-(1-(6-(2-(pyrrolidin-1-yl)ethylamino)pyrimidin-4-yl)-1H-1,2,4-triazol-5-ylamino)benzamide;
N-(3-(1-(6-(2-(dimethylamino)ethylamino)pyrimidin-4-yl)-1H-1,2,4-triazol-5-ylamino)-4-methylphenyl)-3-(trifluoromethyl)benzamide;
N-(2-tert-butylpyridin-4-yl)-3-(1-(6-(2-(dimethylamino)ethylamino)pyrimidin-4-yl)-1H-1,2,4-triazol-5-ylamino)-4-methylbenzamide;
4-methyl-3-(1-(6-(3-(4-methylpiperazin-1-yl)propylamino)pyrimidin-4-yl)-1H-1,2,4-triazol-5-ylamino)-N-(3-(trifluoromethyl)phenyl)benzamide;
N-(2-tert-butylpyridin-4-yl)-4-methyl-3-(1-(6-(3-(4-methylpiperazin-1-yl)propylamino)pyrimidin-4-yl)-1H-1,2,4-triazol-5-ylamino)benzamide;
4-methyl-3-(1-(6-(3-morpholinopropylamino)pyrimidin-4-yl)-1H-1,2,4-triazol-5-ylamino)-N-(3-(trifluoromethyl)phenyl)benzamide;
N-(2-tert-butylpyridin-4-yl)-4-methyl-3-(1-(6-(3-morpholinopropylamino)pyrimidin-4-yl)-1H-1,2,4-triazol-5-ylamino)benzamide;
4-methyl-3-(1-(6-(1-methylpiperidin-4-ylamino)pyrimidin-4-yl)-1H-1,2,4-triazol-5-ylamino)-N-(3-(trifluoromethyl)phenyl)benzamide;
3-(1-(6-(cyclopropylamino)pyrimidin-4-yl)-1H-1,2,4-triazol-5-ylamino)-4-methyl-N-(3-(trifluoromethyl)phenyl)benzamide;
N-(2-tert-butylpyridin-4-yl)-3-(1-(6-(cyclopropylamino)pyrimidin-4-yl)-1H-1,2,4-triazol-5-ylamino)-4-methylbenzamide;
3-(1-(6-(2-methoxyethylamino)pyrimidin-4-yl)-1H-1,2,4-triazol-5-ylamino)-4-methyl-N-(3-(trifluoromethyl)phenyl)benzamide;
N-(2-tert-butylpyridin-4-yl)-3-(1-(6-(2-methoxyethylamino)pyrimidin-4-yl)-1H-1,2,4-triazol-5-ylamino)-4-methylbenzamide;
N-(2-tert-butylpyridin-4-yl)-4-methyl-3-(1-(6-(methylamino)pyrimidin-4-yl)-1H-1,2,4-triazol-5-ylamino)benzamide;
4-methyl-3-(1-(6-(methylamino)pyrimidin-4-yl)-1H-1,2,4-triazol-5-ylamino)-N-(4-(trifluoromethyl)pyridin-2-yl)benzamide;
N-(4-methyl-3-(1-(6-(4-methylpiperazin-1-ylamino)pyrimidin-4-yl)-1H-1,2,4-triazol-5-ylamino)phenyl)-3-(trifluoromethyl)benzamide;
N-(2-tert-butylpyridin-4-yl)-4-methyl-3-(1-(6-(4-methylpiperazin-1-ylamino)pyrimidin-4-yl)-1H-1,2,4-triazol-5-ylamino)benzamide;
N-(4-methyl-3-(1-(6-(2-morpholinoethylamino)pyrimidin-4-yl)-1H-1,2,4-triazol-5-ylamino)phenyl)-3-(trifluoromethyl)benzamide;
N-(2-tert-butylpyridin-4-yl)-4-methyl-3-(1-(6-(2-morpholinoethylamino)pyrimidin-4-yl)-1H-1,2,4-triazol-5-ylamino)benzamide;
4-methyl-3-(1-(6-(4-methylpiperazin-1-ylamino)pyrimidin-4-yl)-1H-1,2,4-triazol-5-ylamino)-N-(4-(trifluoromethyl)pyridin-2-yl)benzamide;
4-methyl-3-(1-(6-(2-morpholinoethylamino)pyrimidin-4-yl)-1H-1,2,4-triazol-5-ylamino)-N-(4-(trifluoromethyl)pyridin-2-yl)benzamide;
4-methyl-3-(1-(6-(4-methylpiperazin-1-ylamino)pyrimidin-4-yl)-1H-1,2,4-triazol-5-ylamino)-N-(3-(trifluoromethyl)phenyl)benzamide;
4-methyl-3-(1-(6-(2-morpholinoethylamino)pyrimidin-4-yl)-1H-1,2,4-triazol-5-ylamino)-N-(3-(trifluoromethyl)phenyl)benzamide;
2-fluoro-N-(4-methyl-3-(1-(6-(4-methylpiperazin-1-ylamino)pyrimidin-4-yl)-1H-1,2,4-triazol-5-ylamino)phenyl)-5-(trifluoromethyl)benzamide;
3-fluoro-N-(4-methyl-3-(1-(6-(4-methylpiperazin-1-ylamino)pyrimidin-4-yl)-1H-1,2,4-triazol-5-ylamino)phenyl)-5-(trifluoromethyl)benzamide;
4-fluoro-N-(4-methyl-3-(1-(6-(4-methylpiperazin-1-ylamino)pyrimidin-4-yl)-1H-1,2,4-triazol-5-ylamino)phenyl)-3-(trifluoromethyl)benzamide;
4-chloro-N-(4-methyl-3-(1-(6-(4-methylpiperazin-1-ylamino)pyrimidin-4-yl)-1H-1,2,4-triazol-5-ylamino)phenyl)-3-(trifluoromethyl)benzamide;
2-tert-butyl-N-(4-methyl-3-(1-(6-(4-methylpiperazin-1-ylamino)pyrimidin-4-yl)-1H-1,2,4-triazol-5-ylamino)phenyl)isonicotinamide;
2-(1,1-difluoroethyl)-N-(4-methyl-3-(1-(6-(4-methylpiperazin-1-ylamino)pyrimidin-4-yl)-1H-1,2,4-triazol-5-ylamino)phenyl)isonicotinamide;
3-(2-cyanopropan-2-yl)-N-(4-methyl-3-(1-(6-(4-methylpiperazin-1-ylamino)pyrimidin-4-yl)-1H-1,2,4-triazol-5-ylamino)phenyl)benzamide;
3-(2-methoxypropan-2-yl)-N-(4-methyl-3-(1-(6-(4-methylpiperazin-1-ylamino)pyrimidin-4-yl)-1H-1,2,4-triazol-5-ylamino)phenyl)benzamide;
2-fluoro-N-(4-methyl-3-(1-(6-(2-morpholinoethylamino)pyrimidin-4-yl)-1H-1,2,4-triazol-5-ylamino)phenyl)-5-(trifluoromethyl)benzamide;
3-fluoro-N-(4-methyl-3-(1-(6-(2-morpholinoethylamino)pyrimidin-4-yl)-1H-1,2,4-triazol-5-ylamino)phenyl)-5-(trifluoromethyl)benzamide;
4-fluoro-N-(4-methyl-3-(1-(6-(2-morpholinoethylamino)pyrimidin-4-yl)-1H-1,2,4-triazol-5-ylamino)phenyl)-3-(trifluoromethyl)benzamide;
4-chloro-N-(4-methyl-3-(1-(6-(2-morpholinoethylamino)pyrimidin-4-yl)-1H-1,2,4-triazol-5-ylamino)phenyl)-3-(trifluoromethyl)benzamide;
2-tert-butyl-N-(4-methyl-3-(1-(6-(2-morpholinoethylamino)pyrimidin-4-yl)-1H-1,2,4-triazol-5-ylamino)phenyl)isonicotinamide;
2-(1,1-difluoroethyl)-N-(4-methyl-3-(1-(6-(2-morpholinoethylamino)pyrimidin-4-yl)-1H-1,2,4-triazol-5-ylamino)phenyl)isonicotinamide;
3-(2-cyanopropan-2-yl)-N-(4-methyl-3-(1-(6-(2-morpholinoethylamino)pyrimidin-4-yl)-1H-1,2,4-triazol-5-ylamino)phenyl)benzamide;
3-(2-methoxypropan-2-yl)-N-(4-methyl-3-(1-(6-(2-morpholinoethylamino)pyrimidin-4-yl)-1H-1,2,4-triazol-5-ylamino)phenyl)benzamide;
3-(4-ethylpiperazin-1-yl)-N-(4-methyl-3-(1-(6-(2-morpholinoethylamino)pyrimidin-4-yl)-1H-1,2,4-triazol-5-ylamino)phenyl)-5-(trifluoromethyl)benzamide;
tert-butyl 4-(3-(4-methyl-3-(1-(6-(2-morpholinoethylamino)pyrimidin-4-yl)-1H-1,2,4-triazol-5-ylamino)phenylcarbamoyl)-5-(trifluoromethyl)phenyl)piperazine-1-carboxylate;
N-(4-methyl-3-(1-(6-(morpholinoamino)pyrimidin-4-yl)-1H-1,2,4-triazol-5-ylamino)phenyl)-3-(trifluoromethyl)benzamide;
4-methyl-3-(1-(6-(morpholinoamino)pyrimidin-4-yl)-1H-1,2,4-triazol-5-ylamino)-N-(3-(trifluoromethyl)phenyl)benzamide;
N-(4-methyl-3-(1-(6-(2-(4-methylpiperazin-1-yl)ethylamino)pyrimidin-4-yl)-1H-1,2,4-triazol-5-ylamino)phenyl)-3-(trifluoromethyl)benzamide;
4-methyl-3-(1-(6-(2-(4-methylpiperazin-1-yl)ethylamino)pyrimidin-4-yl)-1H-1,2,4-triazol-5-ylamino)-N-(3-(trifluoromethyl)phenyl)benzamide;
N-(4-methyl-3-(1-(6-(2-morpholinoethylamino)pyrimidin-4-yl)-1H-1,2,4-triazol-5-ylamino)phenyl)-3-(piperazin-1-yl)-5-(trifluoromethyl)benzamide;
1-(4-chloro-3-(trifluoromethyl)phenyl)-3-(4-methyl-3-(1-(6-(4-methylpiperazin-1-ylamino)pyrimidin-4-yl)-1H-1,2,4-triazol-5-ylamino)phenyl)urea;
1-(4-fluoro-3-(trifluoromethyl)phenyl)-3-(4-methyl-3-(1-(6-(4-methylpiperazin-1-ylamino)pyrimidin-4-yl)-1H-1,2,4-triazol-5-ylamino)phenyl)urea;
N-(4-chloro-3-(trifluoromethyl)phenyl)-4-methyl-3-(1-(6-(2-morpholinoethylamino)pyrimidin-4-yl)-1H-1,2,4-triazol-5-ylamino)benzamide;
N-(4-fluoro-3-(trifluoromethyl)phenyl)-4-methyl-3-(1-(6-(2-morpholinoethylamino)pyrimidin-4-yl)-1H-1,2,4-triazol-5-ylamino)benzamide;
N-(4-methyl-3-(3-(methylamino)-1-(6-(methylamino)pyrimidin-4-yl)-1H-1,2,4-triazol-5-ylamino)phenyl)-3-(trifluoromethyl)benzamide;
1-(4-methyl-3-(1-(6-(methylamino)pyrimidin-4-yl)-1H-1,2,4-triazol-5-ylamino)phenyl)-3-(3-(trifluoromethyl)phenyl)urea;
1-(3-fluoro-5-(trifluoromethyl)phenyl)-3-(4-methyl-3-(1-(6-(methylamino)pyrimidin-4-yl)-1H-1,2,4-triazol-5-ylamino)phenyl)urea;
1-(4-fluoro-3-(trifluoromethyl)phenyl)-3-(4-methyl-3-(1-(6-(methylamino)pyrimidin-4-yl)-1H-1,2,4-triazol-5-ylamino)phenyl)urea;
1-(4-chloro-3-(trifluoromethyl)phenyl)-3-(4-methyl-3-(1-(6-(methylamino)pyrimidin-4-yl)-1H-1,2,4-triazol-5-ylamino)phenyl)urea;
1-(3-(4-ethylpiperazin-1-yl)-5-(trifluoromethyl)phenyl)-3-(4-methyl-3-(1-(6-(methylamino)pyrimidin-4-yl)-1H-1,2,4-triazol-5-ylamino)phenyl)urea;
1-(4-methyl-3-(1-(6-(methylamino)pyrimidin-4-yl)-1H-1,2,4-triazol-5-ylamino)phenyl)-3-(4-((4-methylpiperazin-1-yl)methyl)-3-(trifluoromethyl)phenyl)urea;
N-(3-(2-(dimethylamino)ethoxy)-5-(trifluoromethyl)phenyl)-4-methyl-3-(1-(6-(methylamino)pyrimidin-4-yl)-1H-1,2,4-triazol-5-ylamino)benzamide;
N-(3-(3-(dimethylamino)propoxy)-5-(trifluoromethyl)phenyl)-4-methyl-3-(1-(6-(methylamino)pyrimidin-4-yl)-1H-1,2,4-triazol-5-ylamino)benzamide;
4-methyl-3-(1-(6-(methylamino)pyrimidin-4-yl)-1H-1,2,4-triazol-5-ylamino)-N-(3-(2-(2-oxopyrrolidin-1-yl)ethoxy)-5-(trifluoromethyl)phenyl)benzamide;
4-methyl-3-(1-(6-(methylamino)pyrimidin-4-yl)-1H-1,2,4-triazol-5-ylamino)-N-(3-(2-(pyrrolidin-1-yl)ethoxy)-5-(trifluoromethyl)phenyl)benzamide;
N-(3-(2-(diethylamino)ethoxy)-5-(trifluoromethyl)phenyl)-4-methyl-3-(1-(6-(methylamino)pyrimidin-4-yl)-1H-1,2,4-triazol-5-ylamino)benzamide;
1-(2-fluoro-5-(trifluoromethyl)phenyl)-3-(4-methyl-3-(1-(6-(methylamino)pyrimidin-4-yl)-1H-1,2,4-triazol-5-ylamino)phenyl)urea;
1-(2-chloro-5-(trifluoromethyl)phenyl)-3-(4-methyl-3-(1-(6-(methylamino)pyrimidin-4-yl)-1H-1,2,4-triazol-5-ylamino)phenyl)urea;
1-(3-(4-ethylpiperazin-1-yl)-5-(trifluoromethyl)phenyl)-3-(4-methyl-3-(1-(6-(methylamino)pyrimidin-4-yl)-1H-1,2,4-triazol-5-ylamino)phenyl)urea;
1-(3-((2-(dimethylamino)ethyl)(methyl)amino)-5-(trifluoromethyl)phenyl)-3-(4-methyl-3-(1-(6-(methylamino)pyrimidin-4-yl)-1H-1,2,4-triazol-5-ylamino)phenyl)urea;
1-(3-((2-methoxyethyl)(methyl)amino)-5-(trifluoromethyl)phenyl)-3-(4-methyl-3-(1-(6-(methylamino)pyrimidin-4-yl)-1H-1,2,4-triazol-5-ylamino)phenyl)urea;
1-(3-(2-(dimethylamino)ethoxy)-5-(trifluoromethyl)phenyl)-3-(4-methyl-3-(1-(6-(methylamino)pyrimidin-4-yl)-1H-1,2,4-triazol-5-ylamino)phenyl)urea;
1-(3-((4-ethylpiperazin-1-yl)methyl)-5-(trifluoromethyl)phenyl)-3-(4-methyl-3-(1-(6-(methylamino)pyrimidin-4-yl)-1H-1,2,4-triazol-5-ylamino)phenyl)urea;
N-(3-((dimethylamino)methyl)-5-(trifluoromethyl)phenyl)-4-methyl-3-(1-(6-(methylamino)pyrimidin-4-yl)-1H-1,2,4-triazol-5-ylamino)benzamide; and
3-((3-hydroxyazetidin-1-yl)methyl)-N-(4-methyl-3-(1-(6-(methylamino)pyrimidin-4-yl)-1H-1,2,4-triazol-5-ylamino)phenyl)-5-(trifluoromethyl)benzamide.

14. A pharmaceutical composition comprising a therapeutically effective amount of a compound of claim 1 and a pharmaceutically acceptable carrier.

15. A pharmaceutical composition comprising a therapeutically effective amount of a compound of claim 13 and a pharmaceutically acceptable carrier.

16. A method for treating a cell proliferative disorder, comprising administering to a system or subject in need of such treatment an effective amount of a compound of Formula (1) or a pharmaceutical composition thereof, thereby treating said condition.

17. The method of claim 16, comprising administering said compound to a cell or tissue system; or to a human or animal subject

18. The method of claim 16, wherein said cell proliferative disorder is melanoma, thyroid cancer, colon cancer, cholangiocarcinoma or ovarian cancer.

19. The method of claim 16, wherein said cell proliferative disorder is mediated by abnormal protein kinase activity.

20. A method for treating a B-Raf-mediated condition, comprising administering to a system or subject in need of such treatment an effective amount of a compound of claim 1, or pharmaceutically acceptable salts or pharmaceutical compositions thereof, thereby treating said condition.
